(19) United States
(12) Patent Application Publication
(10) Pub. No.: US 2022/0106562 A1
(43) Pub. Date: Apr. 7, 2022

(54) DIFFERENTIATED AND NONDIFFERENTIATED MSC COMPOSITIONS AND USE THEREOF

(71) Applicant: EXOSTEM BIOTEC LTD., Tel Aviv (IL)

(72) Inventors: Chaya BRODIE, Southfield, MI (US); Aharon BRODIE, Meitar (IL)

(21) Appl. No.: 17/503,443

(22) Filed: Oct. 18, 2021

Related U.S. Application Data
(63) Continuation of application No. PCT/IL2020/050459, filed on Apr. 19, 2020.
(60) Provisional application No. 62/835,557, filed on Apr. 18, 2019.

(51) Int. Cl. C12N 5/079; A61K 35/30; A61P 25/00; A61P 25/16

(52) U.S. Cl. CPC C12N 5/0622; A61K 35/30; A61P 25/00; A61P 25/16; C12N 2501/727; C12N 2500/38; C12N 2501/135; C12N 2501/41; C12N 2501/235; C12N 2501/155; C12N 2506/13; C12N 2501/115; C12N 2501/11; C12N 2501/01

(57) ABSTRACT

Cells with a mixed mesenchymal stem cell (MSC) and astrocyte phenotype are provided. Pharmaceutical compositions comprising these cells, extracellular vesicles from these cells as well as methods of production and methods of use are also provided.

### CROSS REFERENCE TO RELATED APPLICATIONS

This application is a continuation of PCT Patent Application No. PCT/IL2020/050459 filed on Apr. 19, 2020, which claims the benefit of priority of U.S. Provisional Patent Application No. 62/835,557, filed Apr. 18, 2019, all titled “DIFFERENTIATED AND NONDIFFERENTIATED MSC COMPOSITIONS AND USE THEREOF”. The contents of the above applications are all incorporated by reference as if fully set forth herein in their entirety.

### FIELD OF INVENTION

The present invention is in the field of mesenchymal stem cells and extracellular vesicles.

### BACKGROUND OF THE INVENTION

MSCs exert their therapeutic effects in a large number of neurological, inflammatory and degenerative disorders by paracrine effects, via the secretion of cytokines and extracellular vesicles. However, in many cases these broad-spectrum effects are transient and further cannot provide a cure in disorders in which cellular replacement is required. Moreover, the use of unmodified cells exerts general paracrine effects but does not provide specific factors that are required for the treatment of specific disorders. Cell replacement therapy also has numerous hurdles to overcome for full efficacy, not the least of which is rejection of the replacement cells and the limited ability of the replaced cells to function in a hostile environment.

In neurological disorders, there is a non-cell autonomous effect of glial cells that contributes to the pathogenesis of these diseases, regardless of the original cause of pathogenesis. In addition, in many neurological disorders, there are two common factors that contribute to the progression of the disease: accumulation of the neurotransmitter glutamate and inflammatory responses. Lack of neurotrophins, such as BDNF, NGF and GDNF, is also characteristic of many neurological disorders. MSCs can be differentiated into astrocyte-like cells expressing glutamate transporters, glutamine synthase and high levels of BDNF and GDNF (see International Patent Application PCT/IB2013/051430 herein incorporated by reference in its entirety). These astrocyte-like cells can serve as a general therapeutic approach in multiple neurological disorders due to their ability to remove glutamate and degrade it, and their ability to secrete high levels of BDNF.

Recent studies have demonstrated that astrocytes can participate in the neuroinflammation process in the brain. It is known that this process is initially and mainly controlled by microglia and their differentiation into M1 and M2 cells. However, it is now recognized that microglia also affect astrocytes and can induce their differentiation into A1 cells that exert neurotoxic effects by secreting factors such as complement. The conversion of astrocytes into A1 is also evident in degenerative disorders such as ALS and during aging. In contrast, ischemia leads to the differentiation of astrocytes into A2, which exert protective effects. Compositions and methods that harness the beneficial effects of cell replacement and MSC therapy, specifically astrocyte therapy, are greatly needed.

### SUMMARY OF THE INVENTION

The present invention provides cells with a mixed MSC and astrocyte phenotype. Extracellular vesicles from these cells, as well as pharmaceutical compositions comprising these cells are also provided. Methods of producing the cells are provided, as are uses of the cells, vesicles and compositions to treat neurological disorders and diseases and uses of the cells and vesicles in combination with other cells.

According to a first aspect, there is provided a cell comprising mixed mesenchymal stem cell (MSC) and astrocyte (AS) phenotypes (MSC-AS), wherein the cell expresses at least one marker selected from: S100A10, TGM1, PTX3, SPHK1, CD109, Arginase-1, TM4SFL, S1PR3, CLCF1, LCN2, NRF2, prokineticin-2, STAT3 and PKC epsilon.

According to some embodiments, the astrocyte phenotype is an A2 astrocyte phenotype.

According to some embodiments, the cell is resistant to induction to an A1 astrocyte phenotype.

According to some embodiments, the induction comprises stimulation with at least one of C1q, IL-1, TNF-alpha and LPS-induced microglial cells.

According to some embodiments, the cell inhibits the differentiation of astrocytes toward an A1 phenotype.

According to some embodiments, the cell comprises an MSC phenotype comprising at least one of:
a. expression of a plurality of markers selected from the group consisting of: CD73, CD105, CD90, CD146, and CD44 expression and absence of WWII expression;
b. immunosuppression ability;
c. anti-inflammatory ability;
d. the ability to home to sites of inflammation, injury or disease, and
e. expression and/or secretion of neurotrophic factors.

According to another aspect, there is provided a method of producing a cell of mixed MSC and AS phenotypes (MSC-AS), the method comprising at least one of:
a. incubating an MSC or MSC transdifferentiated into a neuronal stem cell (NSC) in low-attachment plates in a first medium and inhibiting GSK3 in the MSC or transdifferentiated MSC; further incubating in a second medium supplemented with retinoic acid, a cAMP activator, and a hedgehog activator; and further incubating in a third medium supplemented with leukemia inhibitory factor (LIF), and Bone morphogenetic protein-4 (BMP4); and
b. incubating an MSC in a first medium supplemented with growth factors in low-attachment plates; further incubating in a second medium comprising serum supplemented with a beta-adrenergic receptor agonist, a neuregulin and growth factors and further incubating in a third medium supplemented with G5, a beta-adrenergic receptor agonist, a neuregulin and growth factors;
thereby producing a hybrid MSC-AS cell.

According to some embodiments, at least one of SOX2 and BRN2 is overexpressed in the MSC transdifferentiated to an NSC before the incubating in a first media.

According to some embodiments, the first media is neurobasal medium or F12 media supplemented with B27.

According to some embodiments, the second media further comprises growth factors.

According to some embodiments, the growth factors are selected from FGF, EGF, PDGF, and FGFbeta.

According to some embodiments, the method further comprises selecting a cell that expresses EAAT1 and/or EAAT2 or secretes a neurotrophic factor selected from BDNF, GDNF, Neurturin, NGF, NT-3, and VEGF.

According to some embodiments, the method further comprised expressing in the MSC or transdifferentiated MSC at least one of: SOX9, NF1A, NF1B, STAT3, miR-21, miR-27, miR-152, miR-455, miR-203, miR-355, let-7, and miR-1.

According to some embodiments, the method further comprises inhibiting in the MSC or transdifferentiated MSC at least one of: miR-224, miR-3191, miR-124, miR-145, miR-1277, miR-107, miR-130, miR-190, miR-1277, miR-190, miR-19, miR-331, combination of miR-124, miR-145 and miR-1277, miR-223, miR-3714, miR-3924, miR-5011, miR-6801, miR-1224, miR-1305, miR-3153, and miR-137.

According to some embodiments, the inhibiting comprises expressing in the MSC or transdifferentiated MSC an RNA that hybridizes to and inhibits the miR.

According to some embodiments, the method further comprises inhibiting in the MSC or transdifferentiated MSC at least one of: SNAIL TWIST1, RUNX2 and SOX11.

According to another aspect, there is provided a cell produced by a method of the invention.

According to another aspect, there is provided extracellular vesicles from a cell of the invention.

According to another aspect, there is provided a pharmaceutical composition comprising at least one of:
a. a cell of the invention;
b. extracellular vesicles of the invention; and
c. conditioned media from a cell of the invention.

According to some embodiments, the pharmaceutical composition further comprises a pharmaceutically acceptable carrier, excipient or adjuvant.

According to another aspect, there is provided a pharmaceutical composition comprising a cell of mixed mesenchymal stem cell (MSC) and astrocyte (AS) phenotype (MSC-AS) and/or exosomes, extracellular vesicles or condition media therefrom, a pharmaceutically acceptable carrier, excipient or adjuvant and at least one of:
a. an undifferentiated MSC;
b. a natural glial cell;
c. a natural neuronal cell;
d. an MSC transdifferentiated to a neuronal cell; and
e. exosomes, extracellular vesicles or conditioned media therefrom.

According to some embodiments, the MSC-AS hybrid cell is a cell of the invention.

According to some embodiments, the neuronal cell is an NSC.

According to some embodiments, the glial cell is an astrocyte.

According to another aspect, there is provided a method of treating a neurological disorder, disease or condition, in a subject in need thereof, the method comprising administering to the subject at least one of:
a. a cell of mixed mesenchymal stem cell (MSC) and astrocyte (AS) phenotype (MSC-AS);
b. exosomes, extracellular vesicles or condition media from the MSC-AS;
c. a chorionic placenta (CH) or umbilical cord (UC) derived MSC; and
d. exosomes, extracellular vesicles or condition media from the CH or UC derived MSC;
thereby treating a neurological disorder, disease or condition.

According to some embodiments, the method further comprises administering to the subject at least one other cell selected from:
a. an undifferentiated MSC;
b. a natural glial cell;
c. a natural neuronal cell; and
d. an MSC transdifferentiated to a neuronal cell.

According to some embodiments, the method comprises administering a pharmaceutical composition of the invention.

According to some embodiments, the MSC-AS, CH MSC, UC MSC or exosomes, extracellular vesicles or condition media therefrom is administered concomitantly, before or after the at least one other cell.

According to some embodiments, the method comprises administering the MSC-AS or exosomes, extracellular vesicles or condition media therefrom.

According to some embodiments, the neurological disorder, disease or condition is selected from: Alzheimer's disease, depression, a psychiatric disorder, dementia, vascular dementia, Lewy body dementia prion disorder, addiction, withdrawal, substance abuse, Amyotrophic lateral sclerosis (ALS), autism, ischemic brain injury, stroke, Parkinson's disease, multiple system atrophy (MSA), multiple sclerosis (MS), Huntingdon's disease, myelin relate disorders, leukodystrophy, cerebrovascular disorders, autism spectrum disorders, attention deficit disorders, prior disease, sleep and circadian disorders, neurological inflammation, encephalopathy, Alexander disease, demyelination disease, brain injury, spinal injury, concussion, radiation-induce brain injury, epilepsy, anesthesia-induced cognitive impairment, aging, neurological aging, chronic pain, infection of the central nervous system (CNS), neuroinflammation and Rett syndrome.

According to some embodiments, the neurological disorder, disease or condition is selected ALS, Parkinson's disease, brain injury, radiation-induced brain injury and ischemic brain injury.

According to some embodiments, the brain injury is selected from traumatic brain injury, stroke, radiation-induced brain injury, ischemic brain injury, prolonged ischemic brain injury, acute radiation induced brain injury, concussion and spaceflight induced brain injury.

According to another aspect, there is provided a pharmaceutical composition of the invention for use in treating a neurological disorder, disease or condition.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

FIG. 1: Bar chart of the A1/A2 ratio in MSC-AS and natural human AS after 48 hours of A1 stimulation. The cells grown with no stimulation were used as control, and the A1/A2 ratio of each cell type without stimulation is normalized to 1. Error bars represent standard error.

FIG. 2: Bar chart of C3 (an A1 marker) expression in MSC-AS cells under various stimuli. Error bars represent standard error.

FIG. 3: Bar chart of the A1/A2 ratio of human astrocytes grown in various conditions and with the addition of MSC conditioned media or MSC exosomes. Astrocytes with a control vector and grown in media were set as a ratio of 1. Error bars represent standard error.

FIG. 4. Bar chart of C3 (an A1 marker) expression in human astrocytes grown in various conditions and with the addition of MSCs or their exosomes. Astrocytes grown alone in media were set as an expression of 1. Error bars represent standard error.

FIG. 5. Bar chart of cell survival, as measured by MTT assay, of motor neurons in transwell culture with mtSOD expressing astrocytes as well as various combinations of MSCs. Error bars represent standard error.

FIG. 6. Bar chart of % cell death in NSC34 cells co-cultured in a transwell dish with mtSOD astrocytes and various MSCs and exosomes. All lanes including MSCs or exosomes show NSC34 cells with mtSOD. Error bars represent standard error. *=a Pval of <0.02.

FIG. 7. Bar chart summarizing protein expression of WT SOD and mtSOD in NSC34 cells treated with MSC and exosomes loaded with an antisense oligonucleotide specific to mutant SOD. Error bars represent standard error. **=a Pval of <0.001.

FIG. 8. Bar chart showing the relative amount of oligodendrocyte differentiation and A1 and A2 astrocyte number in cocultures of astrocytes and OPC treated with CoCl2. Numbers are as compared to a control coculture without CoCl2 addition, which was standardized to 1. Error bars represent standard error.

FIG. 9. Bar chart of relative cell death of neurons in control conditions or after hypoxia+no glucose. Error bars represent standard error. **=a Pval of <0.001.

FIG. 10. Bar chart of relative cell death of neurons in control conditions or after irradiation. Error bars represent standard error. **=a Pval of <0.001, *=a Pval of <0.005.

### DETAILED DESCRIPTION OF THE INVENTION

The present invention, in some embodiments, provides cells with a mixed MSC and astrocyte phenotype. Extracellular vesicles from these cells, as well as pharmaceutical compositions comprising these cells are also provided. Methods of producing the cells are provided, as are uses of the cells, vesicles and compositions to treat neurological disorders and diseases and uses of the cells and vesicles in combination with other cells.

The present invention is based on the surprising finding that mesenchymal stem cells (MSCs) can be transdifferentiated into astrocyte (AS)-like cells that have MSC phenotypes, and specifically A2 astrocyte phenotypes. Further, these cells of mixed phenotype are resistant to acquiring the A1 astrocyte phenotype and even protect other astrocytes from acquiring this deleterious phenotype. This allows for a therapeutic avenue that combines the cell autonomous effects of MSCs and astrocyte cell replacement. It was also demonstrated that these cells alone, and even more so in combination, had positive effects on cells that model neurological disease.

### Cells

By a first aspect, there is provided a cell comprising mixed MSC and AS phenotypes.

In some embodiments, the cell is a mammalian cell. In some embodiments, the cell is a human cell. In some embodiments, the cell is an animal cell. In some embodiments, the animal is a veterinary animal. In some embodiments, the veterinary animal is selected from, a cat, a dog, a horse, a cow, a pig, a sheep and a goat. In some embodiments, the cell is allogenic to a subject in need of treatment for a neurological disease, disorder or condition. In some embodiments, the cell is autologous to a subject in need of treatment for a neurological disease, disorder or condition. In some embodiments, the cell is allogenic to the subject. In some embodiments, the cell is autologous to the subject. In some embodiments, the cell is syngeneic to the subject. In some embodiments, the cell is suspended in appropriate carrier for administration.

As used herein, the term “mesenchymal stem cell” or “MSC”, refers to multipotent stromal cells that have the ability to differentiate into osteoblasts, adipocytes, myocytes, and chondroblasts. MSC are present in bone marrow, adipose tissue, peripheral blood, chorionic placenta, amniotic placenta, amniotic fluid, umbilical cord Wharton's jelly, and dental pulp, among other tissues. The term “multipotent” refers to stem cells which are capable of giving rise to many cell types. In some embodiments, the MSC is derived from umbilical cord or chorionic placenta. In some embodiments, the MSC is derived from dental pulp, umbilical cord or chorionic placenta. In some embodiments, the MSC is derived from chorionic placenta. In some embodiments, the MSC is derived from any one of bone marrow, adipose tissue, peripheral blood, chorionic placenta, amniotic placenta, amniotic fluid, umbilical cord Wharton's jelly, and dental pulp. In some embodiments, the MSC is derived from umbilical cord. In some embodiments, the MSC is derived from dental pulp. In some embodiments, the MSC is derived from any one of umbilical cord and chorionic placenta. In some embodiments, the MSC is derived from any one of amniotic placenta, chorionic placenta, umbilical cord, bone marrow, adipose tissue, and dental pulp.

In some embodiments, the MSC is derived from a stem cell. In some embodiments, the MSC is differentiated from a stem cell. In some embodiments, the stem cell is a naturally occurring stem cell. In some embodiments, the stem cell is a human stem cell. In some embodiments, the stem cell is an adult stem cell. In some embodiments, the stem cell is an embryonic stem cell. In some embodiments, the stem cell is not an embryonic stem cell. In some embodiments, the stem cell is an umbilical cord stem cell. In some embodiments, the stem cell is a placental stem cell. In some embodiments, the stem cell is an induced pluripotent stem cell (iPSC). In some embodiments, the stem cell is a non-naturally occurring stem cell. In some embodiments, the MSC is derived from an iPSC. In some embodiments, MSC is differentiated from an iPSC.

In some embodiments, the MSC is not an amniotic placenta MSCs. In some embodiments, the MSC is not an adipose derived MSC. In some embodiments, a composition of the invention is devoid of amniotic placenta MSCs. In some embodiments, a composition of the invention is devoid of adipose derived MSCs. In some embodiments, a composition of the invention is devoid of an MSC-AS derived from an amniotic placenta MSC. In some embodiments, a composition of the invention is devoid of an MSC-AS derived from an adipose MSC.

Placental, and umbilical cord MSCs, and specifically chorionic placenta MSCs are well known in the art. In some embodiments, these MSCs or their secreted vesicles can be identified by examining the expression of various proteins, and regulatory RNAs such as are described in international patent application WO/2018083700, the content of which are herein incorporated by reference in their entirety. In some embodiments, the MSCs are identified by the tissue they were isolated from. In some embodiments, the MSCs are identified by expression of a marker. In some embodiments, the marker is a protein. In some embodiments, the protein is a surface protein. In some embodiments, the marker is an RNA. In some embodiments, the RNA is an mRNA. In some embodiments, the RNA is a regulatory RNA. In some embodiments, the regulatory RNA is a microRNA (miR). In some embodiments, the marker is a long non-coding RNA (lncRNA). In some embodiments, the marker is a marker provided in WO/2018083700.

Methods of isolating, purifying and expanding mesenchymal stem cells (MSCs) are known in the arts and include, for example, those disclosed by Caplan and Haynesworth in U.S. Pat. No. 5,486,359 and Jones E. A. et al., 2002, Isolation and characterization of bone marrow multipotential mesenchymal progenitor cells, Arthritis Rheum. 46(12): 3349-60.

MSC cultures utilized by some embodiments of the invention preferably include three groups of cells which are defined by their morphological features: small and agranular cells (referred to as RS-1, herein below), small and granular cells (referred to as RS-2, herein below) and large and moderately granular cells (referred to as mature MSCs, herein below). The presence and concentration of such cells in culture can be assayed by identifying a presence or absence of various cell surface markers, by using, for example, immunofluorescence, in situ hybridization, and activity assays.

According to some embodiments, culturing of the mesenchymal stem cells can be performed in any media that support (or at least does not inhibit) the differentiation of the cells towards astrocytic cells such as those described in U.S. Pat. No. 6,528,245 and by Sanchez-Ramos et al. (2000); Woodburry et al. (2000); Woodburry et al. (J. Neurosci. Res. 96:908-917, 2001); Black and Woodbury (Blood Cells Mol. Dis. 27:632-635, 2001); Deng et al. (2001), Kohyama et al. (2001), Reyes and Verfatile (Ann N.Y. Acad. Sci. 938:231-235, 2001) and Jiang et al. (Nature 418:47-49, 2002). The media may be, but is not limited to, F12, G5, neurobasal medium, DMEM, DMEM/F12, OptiMEM™ or any other medium that supports neuronal or astrocytic growth.

In some embodiments, an MSC phenotype comprises anti-inflammation ability. In some embodiments, the MSC or MSC-AS described herein is an anti-inflammatory cell. In some embodiments, an MSC phenotype comprises the ability to decrease inflammation. In some embodiments, an MSC phenotype comprises secretion of anti-inflammatory cytokines. Anti-inflammatory cytokines are well known to one of skill in the art, and include, but are not limited to, IL-10, IL-4, IL-13, and transforming growth factor beta (TGFβ). In some embodiments, an MSC phenotype comprises secretion of neurotrophic factors. As used herein, a “neurotrophic factor” refers to a biomolecule that supports at least one of growth, survival and differentiation of a neuron. In some embodiments, a neurotrophic factor is a peptide. In some embodiments, a neurotrophic factor supports developing neurons. In some embodiments, a neurotrophic factor supports mature neurons. In some embodiments, a neurotrophic factor is selected from BDNF, GDNF, NGF, Neurturin, NT-3 and VEGF. In some embodiments, an MSC phenotype comprises the ability to home to sites of inflammation, injury or disease.

In some embodiments, an MSC phenotype comprises immunomodulation ability. In some embodiments, an MSC phenotype comprises the ability to modulate a subject's immune system. In some embodiments, an MSC phenotype comprises immunosuppression ability. In some embodiments, an MSC phenotype comprises the ability to suppress a subject's immune system. In some embodiments, an MSC phenotype comprises the ability to decrease activation of T-cells.

In some embodiments, an MSC phenotype comprises expression of at least one surface marker selected from the group consisting of: CD73, CD105, CD90, CD44 and CD146. In some embodiments, an MSC phenotype comprises expression of a plurality of surface markers selected from the group consisting of: CD73, CD105, CD90, CD44 and CD146. In some embodiments, an MSC phenotype comprises expression of IL-10. In some embodiments, an MSC phenotype comprises secretion of IL-10. In some embodiments, an MSC phenotype comprises absence of Major Histocompatibility Complex protein II (MHCII) expression. In some embodiments, an MSC phenotype comprises at least one expression marker selected from the group consisting of: CD73, CD105, CD90, CD146, and CD44 expression and absence of MHCII expression. In some embodiments, an MSC phenotype comprises a plurality of expression markers selected from the group consisting of: CD73, CD105, CD90, CD146, and CD44 expression and absence of MHCII expression. In some embodiments, at least one marker is a plurality of markers.

The term “expression” as used herein refers to the biosynthesis of a gene product, including the transcription and/or translation of said gene product. Thus, expression of a nucleic acid molecule may refer to transcription of the nucleic acid fragment (e.g., transcription resulting in mRNA or other functional RNA) and/or translation of RNA into a precursor or mature protein (polypeptide). In some embodiments, expression markers refer to RNA expression. In some embodiments, expression markers refer to protein expression. In some embodiments, surface expression markers refer to expression of proteins on the cell surface or in the plasma membrane of a cell.

Methods of detecting and determining an MSC phenotype are known to one skilled in the art. They include, but are not limited to, staining for MSC surface markers by assays such as FACS or Western Blot. Several commercial kits are available for performing this detecting and determining, including the Human and the Mouse Mesenchymal Stem Cell ID Kits (R&D Systems), MSC Phenotyping Kit, human (Miltenyi Biotech) and the BD Stemflow hMSC Analysis Kit (BD Biosciences). Other methods include measuring secreted pro- and anti-inflammatory cytokines, such as but not limited to IL-1, IL-2, IL-4, IL-10, TNFα, IL-13, and TGFβ, measuring cell homing using homing assays well known in the art and detecting and measuring mRNA expression of MSC transcription factor.

In some embodiments, the MSC and/or its exosomes are allogenic to a subject. In some embodiments, the MSC and/or its exosomes are autologous to a subject. In some embodiments, the MSC and/or its exosomes are semi-autologous. In some embodiments, the MSC and/or its exosomes are syngeneic to a subject. In some embodiments, the MSC and/or its exosomes are allogenic, semi-autologous, syngeneic or autologous to a subject. In some embodiments, the MSC and/or its exosomes do not induce an immune response in a subject. MSC and especially their exosomes and extracellular vesicles have a strong advantage as a therapeutic as they do not express MHCII molecules and do not induce an immune response. Further MSCs and their exosomes actively inhibit the immune response. Chorionic placenta (CH) and umbilical cord (UC) MSCs and their exosomes are particularly effective in this respect. In this way the MSCs and/or their exosomes can be used as an “off the shelf” therapeutic agent that can be administered to any subject in need thereof. The term semi-autologous refers to donor cells which are partially-mismatched to recipient cells at a major histocompatibility complex (MHC) class I or class II locus.

In some embodiments, the cell of the invention is a cell of mixed character. In some embodiments, the cell of the invention is a cell of mixed phenotype. In some embodiments, the cell is an MSC-AS cell. The term MSC-AS is used herein throughout to refer to the cell of the invention. In some embodiments, the cell of the invention is a hybrid cell. As used herein, “hybrid cell” refers to a cell having qualities, characteristics, expression profiles or phenotypes of two different and distinct cell types, for example an MSC and an astrocyte. It does not refer to a physical hybrid in which two separate cells have been made to fuse together. As used here, a hybrid cell is an MSC differentiated toward an astrocyte that has not completed differentiation. In some embodiments, the cell of the invention is a differentiated MSC. In some embodiments, the differentiation is trans-differentiation. In some embodiments, the differentiation is a partial or incomplete differentiation. As used herein, the term “trans-differentiation” refers to differentiation that does not follow a canonical lineage. In some embodiments, trans-differentiation comprises a differentiation that does not occur in nature. In some embodiments, trans-differentiation is differentiation of a cell from one germ layer to a cell from another germ layer.

The term “differentiated MSC” refers to an MSC that have differentiated to possess a specific non-MSC phenotype and expresses markers of that phenotype, but also still retain an MSC phenotype. In some embodiments, a partially differentiated MSC is a cell of a mixed character with both an MSC phenotype and a phenotype of a different cell type. In some embodiments, the other cell type is selected from: a muscle cell, an astrocyte, a neuronal stem cell (NSC), and a differentiated neuron. In some embodiments, the other cell type is selected from: a muscle cell, a glial cell, a neuronal stem cell (NSC), and a differentiated neuron. In some embodiments, the other cell is a glial cell. In some embodiments, the glial cell is an astrocyte. In some embodiments, the differentiated neuron is a motor neuron. In some embodiments, the differentiated neuron is an oligodendrocyte.

Methods of differentiating MSCs are known in the art. In some embodiments, differentiation to an astrocyte phenotype is performed as described in US Application US20150037298. In some embodiments, differentiation to an NSC phenotype or a differentiated neuron phenotype is performed as described in US Application US20150037299. In some embodiments, the method of differentiation to an astrocyte comprises a protocol described hereinbelow. In some embodiments, the protocol is protocol 1 described hereinbelow. In some embodiments, the protocol is protocol 2 described hereinbelow. In some embodiments, the protocol is selected from protocol 1 and protocol 2 described hereinbelow. In some embodiments, an MSC is transdifferentiated to an NSC and then further differentiated to an astrocyte. In some embodiments, the method of differentiation to an NSC comprises a protocol described hereinbelow. In some embodiments, the protocol is protocol 3 described hereinbelow. In some embodiments, the protocol is protocol 4 described hereinbelow. In some embodiments, the protocol is protocol 5 described hereinbelow. In some embodiments, the protocol is selected from protocol 3, protocol 4 and protocol 5 described hereinbelow.

According to some embodiments, the cells has an astrocyte phenotype. Astrocytes are the most abundant type of glial cells in the central nervous system and play major roles in the development and normal physiological functions of the brain. Mature astrocytes can be divided into two types based on their morphology and localization in the brain: fibrous and protoplasmic astrocytes. Fibrous astrocytes populate the white matter and typically have a ‘star-like’ appearance with dense glial filaments that can be stained with the intermediate filament marker glial fibrillary acidic protein (GFAP). Protoplasmic astrocytes are generally found in the grey matter, have more irregular, ‘bushy’, processes and typically have few glial filaments. Astrocytes can regulate water balance, redox potential and ion and neurotransmitter concentrations, secrete neurotrophic factors, remove toxins and debris from the cerebrospinal fluid (CSF) and maintain the blood-brain bather. They also participate in cell-cell signaling by regulating calcium flux, releasing d-serine, producing neuropeptides and modulating synaptic transmission.

In some embodiments, an astrocyte phenotype comprises expression of an astrocyte marker. Examples of astrocyte markers include, but are not limited to: S100 beta, glial fibrillary acidic protein (GFAP), glutamine synthetase, GLT-1, Excitatory Amino Acid Transporter 1 (EAAT1) and Excitatory Amino Acid Transporter 2 (EAAT2). Further, the differentiated cells may secrete a neurotrophic factor including for example glial derived neurotrophic factor (GDNF), GenBank accession nos. L19063, L15306; nerve growth factor (NGF), GenBank accession no. CAA37703; brain-derived neurotrophic factor (BDNF), GenBank accession no CAA62632; neurotrophin-3 (NT-3), GenBank Accession No. M37763; neurotrophin-4/5; Neurturin (NTN), GenBank Accession No. NP-004549; Neurotrophin-4, GenBank Accession No. M86528; Persephin, GenBank accession no. AAC39640; brain derived neurotrophic factor, (BDNF), GenBank accession no. CAA42761; artemin (ART), GenBank accession no. AAD13110; ciliary neurotrophic factor (CNTF), GenBank accession no. NP-000605; insulin growth factor-I (IGF-1), GenBank accession no. NP-000609; and/or Neublastin GenBank accession no. AAD21075. In addition, astrocyte phenotype can be also followed by specific reporters that are tagged with GFP or RFP (or any fluorescent protein) and exhibit increased fluorescence upon astrocyte differentiation. In some embodiments, the marker is a protein marker. In some embodiments, he marker is a gene marker. In some embodiments, the marker is an RNA marker. In some embodiments, an astrocyte marker is selected from S100beta, GFAP, glutamine synthetase, GL T-1, EAAT1 and EAAT2.

In some embodiments, an astrocyte phenotype comprises astrocyte morphology. In some embodiments, an astrocyte phenotype comprises secretion of a neurotrophic factor. In some embodiments, a cell of the invention secretes at least one trophic factor selected from: BDNF, GDNF, Neurturin, NGF, NT-3 and VEGF. In some embodiments, an astrocyte phenotype comprises secretion of an anti-inflammatory cytokine. In some embodiments, an astrocyte phenotype comprises supporting neuronal growth, differentiation and/or health.

In some embodiments, an astrocyte is an A1 or A2 astrocyte. In some embodiments, an astrocyte is an A1 astrocyte. In some embodiments, an astrocyte is an A2 astrocyte. In some embodiments, the astrocyte phenotype is an A2 phenotype. As used herein, an “A1 astrocyte” refers to a neurotoxic astrocyte. As used herein, an “A2 astrocyte” refers to a neuroprotective astrocyte. The A1 and A2 nomenclature parallels the M1 and M2 macrophage nomenclature. In some embodiments, the astrocytes are reactive astrocytes. In some embodiments, an A1 astrocyte phenotype comprises secretion of a proinflammatory cytokine. In some embodiments, an A1 astrocyte phenotype comprises production of reactive oxidation species. In some embodiments, an A2 astrocyte phenotype comprises secretion of an anti-inflammatory cytokine. In some embodiments, an A2 astrocyte phenotype comprises secretion of a neurotrophic factor. In some embodiments, an A2 astrocyte phenotype comprises an immunosuppressive ability. In some embodiments, an A2 astrocyte phenotype comprises secretion of thrombospondins. In some embodiments, an A2 astrocyte phenotype comprises induction of at least one of neuron growth, neuron survival, neuronal differentiation and synapse repair. In some embodiments, an A1 astrocyte phenotype comprises expression of an A1 astrocyte marker. In some embodiments, an A2 astrocyte phenotype comprises expression of an A2 astrocyte marker. In some embodiments, a cell of the invention does not comprise an A1 phenotype. In some embodiments, cell of the invention is not an A1 astrocyte.

According to some embodiments the astrocyte marker is an A1 marker. According to some embodiments, an A1 marker is selected from the group consisting of: Ggta-1 Ggta-1, lipg1, gbp2, Fbln5, Ugt1a, GBP2, Amigo2, C3, H2-T23, Serping1, H2-D1, Gfap1, ligp1, Fkbp5, Psmb8, and Srgn. According to some embodiments, an A1 marker is Ggta-1 Ggta-1, lipg1, gbp2, Fb1n5, Ugt1a, GBP2, Amigo2, C3, H2-T23, Serping1, H2-D1, Gfap1, ligp1, Fkbp5, Psmb8, or Srgn. Each possibility represents a separate embodiment of the invention. According to some embodiments the astrocyte marker is an A2 marker. According to some embodiments, the A2 marker is selected from the group consisting of: Clcf1, Tgm1, Ptx3, S100a10, Sphk1, Cd109, Tm4sf1, SCL10a6, Arginase-1, Nrf2, Prokineticin-2, A2-specific, Ptgs2, Emp1, Slc10a6, B3gnt5, Cd14 and Stat3. According to some embodiments, the A2 marker is Clcf1, Tgm1, Ptx3, S100a10, Sphk1, Cd109, Tm4sf1, SCL10a6, Arginase-1, Nrf2, Prokineticin-2, A2-specific, Ptgs2, Emp1, Slc10a6, B3gnt5, Cd14 or Stat3. Each possibility represents a separate embodiment of the invention. In some embodiments, a marker is a plurality of markers.

According to some embodiments, the cell expresses at least one marker selected from: S100A10A, TGM1, PTX3, SPHK1, CD109, Arginase-1, TM4SF1, S1PR3, CLCF1, LCN2, NRF2, prokineticin-2, STAT3 and PKC epsilon. According to some embodiments, the cell expresses at least one marker selected from: S100A10A, Tgm1, Ptx3, Sphk1, CD109, Arginase-1, Tm4sf1, S1pr3, Clcf1, Lcn2, nrf2 and prokineticin-2, STAT3 and PKC epsilon, GFAP, ALDH1L1, EAAT1, EAAT2, GLAST, BDNF, GDNF, glutamine synthase, GLT-1, IGF-1, CD73, CD105, CD90, CD146, and CD44. In some embodiments, the cell expresses at least one A2 astrocyte marker. According to some embodiments, the cell expresses S100A10A, TGM1, PTX3, SPHK1, CD109, Arginase-1, TM4SF1, S1PR3, CLCF1, LCN2, NRF2, prokineticin-2, STAT3 or PKC epsilon. Each possibility represents a separate embodiment of the invention.

Tissue/cell specific protein markers can be detected using immunological techniques well known in the art, such as those described in Thomson J A et al., (1998) or Science 282: 1145-7. Examples include, but are not limited to, flow cytometry for membrane-bound markers, immunohistochemistry for extracellular and intracellular markers and enzymatic immunoassay, for secreted molecular markers. Gene expression can also be used to detect gene/RNA markers; methods include RT-PCR, qPCR, real-time PCR, northern blot, in situ hybridization, and microarray.

In some embodiments, the cell is resistant to induction of an A1 astrocyte phenotype. In some embodiments, the cell is blocked from induction of an A1 astrocyte phenotype. In some embodiments, the cell comprises reduced induction of an A1 astrocyte phenotype. In some embodiments, the reduction is as compared to a naturally occurring astrocyte. In some embodiments, the reduction is as compared to MSCs differentiated to astrocytes known in the art. In some embodiments, induction of an A1 phenotype comprises conversion to an A1 astrocyte. In some embodiments, induction comprises conversion. In some embodiments, the induction is induction caused by an A1 stimulus. In some embodiments, the induction comprises an A1 stimulus. In some embodiments, the induction comprises stimulation by at least one A1 stimulus. In some embodiments, an A1 stimulus is selected from C1q, IL-1, TNF-alpha and LPS-induced microglial cells. In some embodiments, the A1 stimulus is contact with C1q, IL-1 and/or TNF-alpha. In some embodiments, the A1 stimulus is co-culture or contact with LPS-stimulated microglial cells.

In some embodiments, the cell inhibits the differentiation of an astrocyte toward an A1 phenotype. In some embodiments, the cell inhibits induction of an A1 phenotype in an astrocyte. In some embodiments, the astrocyte is not a cell of the invention. In some embodiments, the cell protects an astrocyte from A1 conversion. In some embodiments, the astrocyte is an astrocyte other than the cell of the invention. In some embodiments, the astrocyte is another cell. In some embodiments, the astrocyte is a natural astrocyte. In some embodiments, the astrocyte is a cell differentiated to an astrocyte. In some embodiments, the differentiation is in vitro differentiation. In some embodiments, the differentiation is trans-differentiation. In some embodiments, the differentiation is a non-natural differentiation. In some embodiments, the astrocyte is a non-active astrocyte. In some embodiments, the astrocyte is an astrocyte that is not committed to and A1 or A2 phenotype. In some embodiments, inhibition is a decrease of at least 10, 20, 25, 30, 40, 50, 60, 70, 75, 80, 90, 95, 97, 99 or 100%. Each possibility represents a separate aspect of the invention. In some embodiments, the decrease is as compared to what occurs in the absence of the cell of the invention. In some embodiments, the decrease is as compared to induction in the absence of the cell of the invention.

In some embodiments, the inhibition of A1 phenotype in another cell is via secreted vesicles. In some embodiments, the inhibition of A1 phenotype in another cell is via exosomes. In some embodiments, the inhibition of A1 phenotype in another cell is via cultured media. In some embodiments, the cell of the invention exerts its effect without cellular contact. In some embodiments, the cell of the invention exerts its effect via cellular contact. In some embodiments, extracellular vesicles and/or conditioned media from a cell of the invention exerts the same effect as the cell itself. In some embodiments, the cell of the invention exerts its effect by direct cellular contact and without cellular contact.

As used herein, “conditioned media” refers to old media that had been on growing cells for at least 1 day. Such media contains secreted factors from the growing cells, such as, but not limited to soluble factors, exosomes, microsomes, and other extracellular vesicles. In some embodiments, the conditioned media had been on growing cells for at least 24, 48, 72, 96 or 120 hours. Each possibility represents a separate embodiment of the invention.

By another aspect, there is provided a cell produced by a method of the invention.

In addition to their use as a therapeutic themselves, the MSC-AS and their vesicles as well as undifferentiated MSCs and their vesicles can be loaded with RNA and peptide-based therapies as well. These include but are not limited to anti-sense oligonucleotides directed against mutant SOD or other mutated proteins, siRNAs targeting specific genes that play a role in neurodegeneration, neuroinflammation and brain injury, miRNAs that are deregulated in these diseases, artificial miRNAs that target specific mutation and modified mRNAs. Exosomes can carry small peptides or chemical and can deliver gene therapy by delivering Crispr/Cas9, viral vectors or other modes of gene therapy. The combination of cells/vesicles that exert a therapeutic effects and RNA or peptide-based therapies can exert a synergistic effect. In some embodiments, the cell of the invention comprises a therapeutic. In some embodiments, the therapeutic is an RNA based therapeutic. In some embodiments, the therapeutic is a peptide therapeutic. In some embodiments, the therapeutic is a drug. In some embodiments, the therapeutic is secreted from the cell. In some embodiments, the secretion is within extracellular vesicles.

The cells and extracellular vesicles of the invention can also be targeted to astrocytes, microglia, neurons or oligodendrocytes via surface expression of targeting moieties. These vesicles can cross the blood-brain barrier (BBB), and can be targeted to the BBB as well. They can also be targeted to sites of injury, damage, and/or disease. In some embodiments, the cell and/or extracellular vesicle from the cell comprise a targeting moiety. In some embodiments, the moiety targets to a glial cell. In some embodiments, the targeting moiety is to a neuronal cell. In some embodiments, the moiety targets to inflammation. In some embodiments, the moiety targets to a site of disease. In some embodiments, the moiety targets to a site of damage. In some embodiments, the moiety targets to the central nervous system (CNS). In some embodiments, the moiety targets to the brain. In some embodiments, the moiety targets to the BBB. In some embodiments, the moiety targets to the spinal cord. In some embodiments, the moiety targets to specific regions of the brain.

### Extracellular Vesicles

By another aspect, there is provided extracellular vesicles from a cell of the invention.

By another aspect, there is provided extracellular vesicles that inhibit the differentiation of an astrocyte toward an A1 phenotype.

In some embodiments, the extracellular vesicles are from a cell. In some embodiments, the cell is a plant cell. In some embodiments, the cell is an animal cell. In some embodiments, the cell is a mammalian cell. In some embodiments, the cell is a human cell. In some embodiments, the cell is an MSC. In some embodiments, the cell is a cell of the invention. In some embodiments, the cell is an MSC-AS. In some embodiments, the inhibiting comprises contact of the exosome with the cell.

The term “extracellular vesicles”, as used herein, refers to all cell-derived vesicles secreted from cells including but not limited to exosomes and microvesicles. In some embodiments, the extracellular vesicles are exosomes. “Exosome”, as used herein, refers to cell-derived vesicles of endocytic origin, with a size of 50-100 nm, and secreted from cells. As a non-limiting embodiment, for the generation of exosomes, cells are maintained with Opti-MEM and human serum albumin or 5% FBS that was depleted from exosomes. In some embodiments, exosomes comprise all extracellular vesicles. “Microvesicles”, as used herein, refers to cell-derived vesicles originating from the plasma membrane, with a size of 100-1000 nm, and secreted from cells. In some embodiments, the extracellular vesicles are fresh. In some embodiments, the extracellular vesicles are frozen. In some embodiments, the extracellular vesicles are lyophilized. In some embodiments, the extracellular vesicles are in culture media. In some embodiments, the extracellular vesicles are configured for administration to a subject.

Exosomes, extracellular vesicles, or microvesicles can be obtained by growing MSCs in culture medium with serum depleted from exosomes or in serum-free media such as OptiMeM and subsequently isolating the exosomes by ultracentrifugation. Other methods associated with beads, columns, filters and antibodies are also employed. In some embodiments, the cells are grown in hypoxic conditions or incubated in medium with low pH so as to increase the yield of the exosomes. In other embodiments, the cells are exposed to radiation so as to increases exosome secretion and yield. In some embodiments, the exosomes are suspended in appropriate carrier for administration. Therapeutic agents can be added directly to the extracellular vesicles or can be expressed in the cell so that they are secreted in the extracellular vesicles.

### Pharmaceutical Compositions

By another aspect, there is provided a pharmaceutical composition comprising at least one of: a cell of the invention, extracellular vesicles of the invention, and conditioned media from a cell of the invention.

In some embodiments, the pharmaceutical composition comprises a cell of the invention. In some embodiments, the pharmaceutical composition comprises conditioned media from a cell of the invention. In some embodiments, the pharmaceutical composition comprises extracellular vesicles of the invention. In some embodiments, the pharmaceutical composition comprises a pharmaceutically acceptable carrier, excipient, or adjuvant.

As used herein, the term “carrier,” “excipient,” or “adjuvant” refers to any component of a pharmaceutical composition that is not the active agent. As used herein, the term “pharmaceutically acceptable carrier” refers to non-toxic, inert solid, semi-solid liquid filler, diluent, encapsulating material, formulation auxiliary of any type, or simply a sterile aqueous medium, such as saline. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide,” U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21st Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles which increase the half-life of the peptides or polypeptides in serum. Liposomes include emulsions, foams, micelies, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. The selection of lipids is generally determined by considerations such as liposome size and stability in the blood. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein. In some embodiments, the pharmaceutical composition comprises a therapeutically effective amount of cells, vesicles and/or media.

The “pharmaceutically effective amount” and/or “therapeutically effective amount” for purposes herein is thus determined by such considerations as are known in the art. The amount must be effective to achieve improvement including but not limited to improved survival rate or more rapid recovery, or improvement or elimination of symptoms and other indicators as are selected as appropriate measures by those skilled in the art.

Administration can by injection to any desired site on the body. However, other methods of administration can also be used, such as transplantation or transfusion with or without specific scaffolds. The dose can be determined by one skilled in the art, such as 0.1×106 cells/kg to 5×106 cells/kg, or 0.1-1 μg of purified exosomes. The MSCs can be harvested from any origin by methods known in the art or by methods described herein. The MSC may be maintained under specific conditions to have the expression profile of the MSC subpopulation as described herein.

It should be noted that MSCs and their exosomes can be administered as the composition and can be administered alone or as an active ingredient in combination with pharmaceutically acceptable carriers, diluents, adjuvants, and vehicles. The composition can also be administered systemically, orally, subcutaneously, or parenterally including intravenous, intraarterial, intramuscular, intraperitoneally, intratonsillar, and intranasal administration as well as intrathecal and infusion techniques. Implants of the compositions are also useful. The patient being treated is a warm-blooded animal and, in particular, mammals including man. The pharmaceutically acceptable carriers, diluents, adjuvants, and vehicles as well as implant carriers generally refer to inert, non-toxic solid or liquid fillers, diluents or encapsulating material not reacting with the active ingredients of the invention. In some embodiments, the pharmaceutical composition is configured for the administration. In some embodiments, the pharmaceutical composition is configured for administration to a subject. In some embodiments, the pharmaceutical composition is configured for systemic administration. In some embodiments, the pharmaceutical composition is configured for local administration. In some embodiments, the pharmaceutical composition is configured for a mode of administration described hereinabove.

The doses can be single doses or multiple doses over a period of several days, weeks, months or even years. The treatment generally has a length proportional to the length of the disease process and drug effectiveness and the patient species being treated.

When administering the composition of the present invention parenterally, it will generally be formulated in a unit dosage injectable form (solution, suspension, emulsion). The pharmaceutical formulations suitable for injection include sterile aqueous solutions or dispersions and sterile powders for reconstitution into sterile injectable solutions or dispersions. The carrier can be a solvent or dispersing medium containing, for example, water, ethanol, polyol (for example, glycerol, propylene glycol, liquid polyethylene glycol, and the like), suitable mixtures thereof, and vegetable oils.

Proper fluidity can be maintained, for example, by the use of a coating such as lecithin, by the maintenance of the required particle size in the case of dispersion and by the use of surfactants. Non-aqueous vehicles such a cottonseed oil, sesame oil, olive oil, soybean oil, corn oil, sunflower oil, or peanut oil and esters, such as isopropyl myristate, may also be used as solvent systems for compound compositions. Additionally, various additives which enhance the stability, sterility, and isotonicity of the compositions, including antimicrobial preservatives, antioxidants, chelating agents, and buffers, can be added. Prevention of the action of microorganisms can be ensured by various antibacterial and antifungal agents, for example, parabens, chlorobutanol, phenol, sorbic acid, and the like. In many cases, it will be desirable to include isotonic agents, for example, sugars, sodium chloride, and the like. Prolonged absorption of the injectable pharmaceutical form can be brought about by the use of agents delaying absorption, for example, aluminum monostearate and gelatin. According to the present invention, however, any vehicle, diluent, or additive used would have to be compatible with the compounds.

Sterile injectable solutions can be prepared by incorporating the compounds utilized in practicing the present invention in the required amount of the appropriate solvent with various of the other ingredients, as desired.

A pharmacological formulation of the present invention can be administered to the patient in an injectable formulation containing any compatible carrier, such as various vehicle, adjuvants, additives, and diluents; or the compounds utilized in the present invention can be administered parenterally to the patient in the form of slow-release subcutaneous implants or targeted delivery systems such as monoclonal antibodies, vectored delivery, iontophoretic, polymer matrices, liposomes, and microspheres. Examples of delivery systems useful in the present invention include: U.S. Pat. Nos. 5,225,182; 5,169,383; 5,167,616; 4,959,217; 4,925,678; 4,487,603; 4,486,194; 4,447,233; 4,447,224; 4,439,196; and 4,475,196. Many other such implants, delivery systems, and modules are well known to those skilled in the art.

In some embodiments, the pharmaceutical composition further comprises at least one of: an undifferentiated MSC, a natural glial cell, a natural neuronal cell, a trans-differentiated MSC and extracellular vesicles or conditioned media from one of these cells. In some embodiments, the pharmaceutical composition further comprises at least one of: an undifferentiated MSC, a natural glial cell, a natural neuronal cell, and a trans-differentiated MSC. In some embodiments, the pharmaceutical composition further comprises an undifferentiated MSC. In some embodiments, the pharmaceutical composition further comprises a natural glial cell. In some embodiments, the pharmaceutical composition further comprises a natural neuronal cell. In some embodiments, the pharmaceutical composition further comprises a transdifferentiated MSC. In some embodiments, the MSC is transdifferentiated to a neuronal cell.

By another aspect, there is provided a pharmaceutical composition comprising an MSC and a glial cell.

By another aspect, there is provided a pharmaceutical composition comprising an MSC and a neuronal cell.

In some embodiments, the MSC is an undifferentiated MSC. In some embodiments, the MSC is a differentiated MSC. In some embodiments, the MSC comprises an MSC phenotype. In some embodiments, the MSC is an MSC of the invention. In some embodiments, the MSC is a cell of mixed MSC and astrocyte phenotype. In some embodiments, the MSC is an MSC-AS. In some embodiments, the glial cell is a natural glial cell. In some embodiments, the neuronal cell is a natural neuronal cell. In some embodiments, the glial cell is a glial cell differentiation from a different cell. In some embodiments, the neuronal cell is a neuronal cell differentiated form a different cell. In some embodiments, the different cell is an MSC. In some embodiments, the different cell is an induced pluripotent stem cell (iPSC).

As used herein, the term “natural” refers to a cell that has not be modified. In some embodiments, the modification is genetic modification. In some embodiments, the modification is differentiation. In some embodiments, a natural cell is a primary cell. In some embodiments, a natural cell is a cell harvested from a subject. In some embodiments, a natural cell is not a cell derived from another cell in culture. In some embodiments, a natural cell is not a cell differentiated from a cell of a different cell type in culture. In some embodiments, a natural cell includes cells expanded from a natural cell wherein the expansion does not comprise differentiation. In some embodiments, a natural cell has been in culture. In some embodiments, a natural cell has not been in culture. In some embodiments, a natural cell is an isolated natural cell. In some embodiments, a natural cell is a cell with only its natural phenotype. In some embodiments, a natural cell is a cell that is not derived from an MSC that has been differentiated to that cell type. In some embodiments, a natural cell is a cell that has not been manipulated. In some embodiments, a natural cell is a cell that underwent natural differentiation. In some embodiments, a natural cell is a cell harvested from a subject. In some embodiments, manipulation does not comprise harvesting or isolating the cell. In some embodiments, a natural cell is a cell that is unmodified. In some embodiments, a natural cell is not a transdifferentiated cell.

In some embodiments, a glial cell is an astrocyte. In some embodiments, a glial cell is selected from an oligodendrocyte, an astrocyte, microglia, a Schwann cell, a satellite cell and an ependymal cell. In some embodiments, a neuronal cell is a neuronal stem cell (NSC). In some embodiments, a neuronal cell is a motor neuron. In some embodiments, a neuronal cell is selected from an NSC, a motor neuron, a sensory neuron, and an interneuron.

In some embodiments, the ratio of MSC to other cell is at least 100:1, 50:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:50 or 1:100. Each possibility represents a separate embodiment of the invention. In some embodiments, the ratio of MSC-AC to other cell is at most 100:1, 50:1, 25:1, 20:1, 15:1, 10:1, 9:1, 8:1, 7:1, 6:1, 5:1, 4:1, 3:1, 2:1, 1:1, 1:2, 1:3, 1:4, 1:5, 1:6, 1:7, 1:8, 1:9, 1:10, 1:15, 1:20, 1:25, 1:50 or 1:100. Each possibility represents a separate embodiment of the invention. In some embodiments, the ration of MSC-AC to other cell is in a range between the above enumerated minimums and maximums.

In some embodiments, one of the cells comprises a therapeutic agent. In some embodiments, both of the cells comprise therapeutic agents. In some embodiments, the extracellular vesicles of an MSC comprise a therapeutic agent. In some embodiments, the extracellular vesicles of an MSC-AS comprise a therapeutic agent. In some embodiments, one of the cells comprises a targeting moiety. In some embodiments, both of the cells comprise targeting moieties. In some embodiments, the extracellular vesicles form one or both cells comprise targeting moieties.

In some embodiments, the pharmaceutical composition of the invention is for use in treating a neurological disease, disorder, or condition. In some embodiments, the cell of the invention is for use in treating a neurological disease, disorder, or condition. In some embodiments, the extracellular vesicles of the invention are for use in treating a neurological disease, disorder, or condition.

### Methods of Production

By another aspect, there is provided a method of producing a cell of mixed MSC and astrocyte phenotypes.

In some embodiments, a cell of mixed MSC and astrocyte phenotypes is a cell of the invention. In some embodiments, a cell of mixed MSC and astrocyte phenotypes is an MSC-AS. In some embodiments, the method is performed in vitro. In some embodiments, the method is performed ex vivo. In some embodiments, the method is performed in culture. In some embodiments, the method is not performed in a subject. In some embodiments, the method is protocol 1 as described hereinbelow. In some embodiments, the method is protocol 2 as described hereinbelow. In some embodiments, the method is selected from protocol 1 and protocol 2 as described hereinbelow.

In some embodiments, the method comprises incubating an MSC in a low-attachment plate in a first media and inhibiting glucose 6-phosphate kinase 3 (GSK3) in said MSC. In some embodiments, inhibiting GSK3 comprises supplementing the first media with a GSK3 inhibitor. In some embodiments, the GSK3 inhibitor is CHIR99021. In some embodiments, the method comprises incubating an MSC in a low-attachment plate in a first media supplemented with a CHIR99021. Examples of GSK3 inhibitors include, but are not limited to, lithium ions, valproic acid, curcumin, CHIR99021 and alanzapine. In some embodiments, the first medium is supplemented with a growth factor.

In some embodiments, the MSC is an MSC trans-differentiated into a neuron. In some embodiments, the MSC is an MSC trans-differentiated into an NSC. In some embodiments, the trans-differentiation is a partial differentiation. In some embodiments, the MSC has a mix of MSC and NSC phenotypes. In some embodiments, the method further comprises transdifferentiating an MSC to a neuronal phenotype or a neuron before the first incubation. In some embodiments, the method of transdifferentiating comprises the protocol of protocol 3 as described hereinbelow. In some embodiments, the method of transdifferentiating comprises the protocol of protocol 4 as described hereinbelow. In some embodiments, the method of transdifferentiating comprises the protocol of protocol 5 as described hereinbelow. In some embodiments, the method of transdifferentiating comprises the protocol of any one of protocol 3 and 4 as described hereinbelow. In some embodiments, the method of transdifferentiating comprises the protocol of any one of protocol 3, 4 and 5 as described hereinbelow.

In some embodiments, the method further comprises incubating the MSC is a second medium. In some embodiments, the first medium is removed, and a second medium is added. In some embodiments, the MSC is washed between. In some embodiments, the MSC is not washed. In some embodiments, the wash is with a salt buffer. In some embodiments, the salt buffer is PBS. In some embodiments, the MSCs are isolated and re-plated before the second medium is added. In some embodiments, the MSCs are still in low attachment plates. In some embodiments, the entire method is performed in low attachment plates. In some embodiments, the second medium is supplemented with retinoic acid (RA). In some embodiments, the RA is all trans-RA. In some embodiments, the second medium is supplemented with a cAMP activator. In some embodiments, the second medium is supplemented with a hedgehog activator. In some embodiments, the second medium is supplemented with growth factors. In some embodiments, the cAMP activator is dcAMP. Examples of cAMP activators are well known in the art and include, but are not limited to dcAMP, forskolin, pituitary adenylate cyclase activating polypeptide 38 and NB001. In some embodiments, the hedgehog activator is purmorphamine. In some embodiments, the hedgehog activator is a smoothened agonist. Examples of hedgehog activators are well known in the art and include, but are not limited to purmorphamine, 20(S)-hydroxycholesterol, SAG and SAG21k. In some embodiments, the growth factor is selected from bFGF, EGF, FGF, FGFbeta, PDGF, FGF2 and a combination thereof. In some embodiments, the second medium is supplemented with bFGF and FGF2.

In some embodiments, the method further comprises incubating the MSC in a third medium. In some embodiments, the third medium is supplemented with leukemia inhibitory factor (LIF). In some embodiments, the third medium is supplemented with a bone morphogenic protein (BMP). In some embodiments, the BMP is BMP4. In some embodiments, the third medium is supplemented with a growth factor. In some embodiments, the MSC is washed between the second and third media. In some embodiments, the MSC is not washed between the second and third media. In some embodiments, the third media is addition of LIF and/or BMP to the second media.

In some embodiments, the incubating is for at least 3, 4, 5, 6, 8, 12, 16, 18 hours, 1, 2, 3, 4, 5, 6, 7, 8, 9, 10, 11, 12, 13, or 14 days. Each possibility represents a separate embodiment of the invention. In some embodiments, the incubating is for at most 1, 2, 3, 4, 5, 6, 7, 8, 9, 10, 11, 12, 13, 14, 15, 16, 17, 18, 19, 20, 21, 22, 23, 24, or 25 days. Each possibility represents a separate embodiment of the invention. In some embodiments, the incubating is for between 1-21, 1-14, 1-10, 1-7, 3-21, 3-14, 3-10, 3-7, 5-21, 5-14, 5-10, 5-7, 7-21, 7-14 or 7-10 days. Each possibility represents a separate embodiment of the invention. In some embodiments, the incubation in the first media is for between 8-12 days, 9-11 days, 8-11 days or 9-12 days. Each possibility represents a separate embodiment of the invention. In some embodiments, the incubation in the second media is for about 10 days. In some embodiments, incubation in the second media is for between 8-12 days, 9-11 days, 8-11 days or 9-12 days. Each possibility represents a separate embodiment of the invention. In some embodiments, the third incubation is for 7-10 days. In some embodiments, the incubation in the third media is for 7-10, 7-11, 7-12, 6-10, 6-11, 6-12, 8-10, 8-11, 8-12 or 8-13 days. Each possibility represents a separate embodiment of the invention. In some embodiments, the incubation in the fourth media is for 7-10, 7-11, 7-12, 6-10, 6-11, 6-12, 8-10, 8-11, 8-12 or 8-13 days. Each possibility represents a separate embodiment of the invention. In some embodiments, incubation in the fifth media is for 7-10, 7-11, 7-12, 6-10, 6-11, 6-12, 8-10, 8-11, 8-12 or 8-13 days. Each possibility represents a separate embodiment of the invention.

In some embodiments, the method comprises exposing an MSC to acidic conditions. In some embodiments, the acidic conditions are a pH of about 6. In some embodiments, acidic conditions are a pH of between 6.5 and 5, 6.5 and 5.5, 6.5 and 5.75, 6.5 and 6, 6.25 and 5, 6.25 and 5.5, 6.25 and 5.75, 6.25 and 6, 6 and 5.5, or 6 and 5.75. Each possibility represents a separate embodiment of the invention. In some embodiments, the exposure is for about an hour. In some embodiments, the exposure is for 30-90, 30-80, 30-70, 30-65, 30-60, 30-50, 40-90, 40-80, 40-70, 40-65, 40-60, 40-55, 45-90, 45-80, 45-70, 45-65, 45-60, 45-50, 50-90, 50-80, 50-70, 50-65, 50-60, 50-55, 55-90, 55-80, 55-70, 55-65, 55-60, 60-90, 60-80, 60-70, or 60-65 minutes. Each possibility represents a separate embodiment of the invention.

In some embodiments, the method further comprises exposing the MSC to hypoxia. In some embodiments, hypoxia comprises an oxygen level at or below 5, 4, 3, 2, 1.5, 1, 0.5, 0.25, or 0.1%. Each possibility represents a separate embodiment of the invention. In some embodiments, the exposure is for overnight. In some embodiments, the exposure is for between 8-36, 8-24, 8-18, 8-16, 8-12, 8-10, 10-36, 10-24, 10-18, 10-16, 10-12, 12-36, 12-24, 12-18 or 12-16 hours. Each possibility represents a separate embodiment of the invention.

In some embodiments, the MSCs are cultured/maintained in media following hypoxia. In some embodiments, the MSC is maintained in MSC media. In some embodiments, the MSC is maintained in F12 media. In some embodiments, the MSC is maintained in B27 supplemented media. In some embodiments, the MSC is maintained in F12+B27 media. In some embodiments, the medium is DMEM. In some embodiments, the medium comprises serum. In some embodiments, the medium is serum free. In some embodiments, the MSC is maintained in NSC media. In some embodiments, the MSC is maintained in astrocyte media. In some embodiments, the media is MSC media. In some embodiments, the media is astrocyte cell media. In some embodiments, the media is stem cell media. Such medias are well known in the art, and include but are not limited to, Astrocyte Medium (Sciencell), Astrocyte Medium (Thermo Fisher), MesenPRO RS Medium (ThermoFisher), StemPro MSC SFM (ThermoFisher), and NutriStem MSC XF Medium (Biological Industries). In some embodiments, the media comprises growth factors. In some embodiments, the growth factors are selected from FGF, EGF and both.

In some embodiments, the method further comprises incubation in a fourth medium comprising serum. In some embodiments, the fourth medium is supplemented with a beta-adrenergic receptor agonist. In some embodiments, the beta-adrenergic receptor agonist albuterol. Beta-adrenergic receptor agonists are well known in the art and include, but are not limited to albuterol, epinephrine, norepinephrine, buphenine, dopexamine, fenoterol, isoetarine, levosalbutamol, and terbutaline. In some embodiments, the fourth medium is supplemented with growth factors. In some embodiments, the growth factors selected from FGFbeta, PDGF and both. In some embodiments, the fourth medium is supplemented with neuregulin. In some embodiments, the MSC is washed between the third and fourth media. In some embodiments, the MSC is not washed between the third and fourth media.

In some embodiments, the method further comprises incubation in a fifth medium. In some embodiments, the fifth medium does not comprise serum. In some embodiments, the fifth medium is supplemented with G5. In some embodiments, the fifth medium is supplemented with a beta-adrenergic receptor agonist. In some embodiments, the firth medium is supplemented with a growth factor. In some embodiments, the growth factor is FGF. In some embodiments, the fifth medium is supplemented with neuregulin. In some embodiments, the MSC is washed between the fourth and fifth media. In some embodiments, the MSC is not washed between the fourth and fifth media.

In some embodiments, a wash is performed between incubations or exposures. In some embodiments, a wash is nor performed. In some embodiments, the MSC is isolated between incubations or exposures. A skilled artisan will appreciate that not all the steps enumerated hereinabove must be performed with each trans-differentiation but rather various combinations of the above may be employed.

In some embodiments, the method further comprises selecting a cell that expressed an astrocyte marker. In some embodiments, the astrocyte marker is an A2 marker. In some embodiments, the marker is a marker of a cell of the invention. In some embodiments, the marker is selected from EAAT1, EAAT2, and secretion of a neurotrophic factor selected from BDNF, GDNF, NGF, NT-3, and VEGF.

In some embodiments, the method further comprises expressing in the MSC at least one of: SOX9, NF1A, NF1B, STAT3, miR-21, miR-27, miR-152, miR-455, miR-203, miR-355, let-7, and miR-1. In some embodiments, the method further comprises inhibiting in the MSC at least miR selected from: miR-224, miR-3191, miR-124, miR-145, miR-1277, miR-107, miR-130, miR-190, miR-1277, miR-190, miR-19, miR-331, combination of miR-124, miR-145 and miR-1277, miR-223, miR-3714, miR-3924, miR-5011, miR-6801, miR-1224, miR-1305, miR-3153, and miR-137. In some embodiments, the inhibiting comprises expressing in the MSC an RNA molecule that hybridizes to and inhibits the at least one miR. In some embodiments, the RNA molecule is a synthetic RNA molecule. In some embodiments, the RNA molecule is an antagomir. In some embodiments, the inhibiting comprises genetic alteration of the MSC. Genetic alteration such as by CRISPR/Cas9, or TALON for example is well known in the art. Any method known in the art for inhibiting or decreasing miR function may be employed, including but not limited to antagomirs, gene editing, and RNA sponge. In some embodiments, the method further comprises inhibiting in the MSC at least one of SNAIL TWIST1, RUNX2 and SOX11.

By another aspect, there is provided a method for trans-differentiation of an MSC to an NSC, the method comprising a protocol selected from protocol 3, protocol 4, protocol 5 and a combination thereof.

In some embodiments, the protocol is protocol 3. In some embodiments, the protocol is protocol 4. In some embodiments, the protocol is protocol 5. In some embodiments, the protocol is selected from protocol 3 and protocol 4.

In some embodiments, an MSC is trans-differentiated to an NSC by a method of the invention and then the MSC-NSC is transdifferentiated to an MSC-AS by a method of the invention.

The conditions used for contacting the mesenchymal stem cells are selected for a time period/concentration of cells/concentration of miRNA/ratio between cells and miRNA which enable the miRNA (or inhibitors thereof) to induce differentiation thereof. The present invention further contemplates incubation of the mesenchymal stem cells with a differentiation factor which promotes differentiation towards an astrocytic lineage. The incubation with such differentiation factors may be affected prior to, concomitant with or following the contacting with the miRNA. According to this embodiment the medium may be supplemented with at least one of B27, SHE (e.g. about 250 ng/ml), FGFb (e.g. 50 ng/ml), EGF (e.g. about 50 ng/ml), a cAMP inducer (e.g. IBMX or dbcycAMP), PDGF (e.g. about 5 ng/ml) neuregulin (e.g. about 50 ng/ml) and FGFb (e.g. about 20 ng/ml).

The term “microRNA”, “miRNA”, and “miR” are synonymous and refer to a collection of non-coding single-stranded RNA molecules of about 19-28 nucleotides in length, which regulate gene expression. MiRNAs are found in a wide range of organisms and have been shown to play a role in development, homeostasis, and disease etiology.

Genes coding for miRNAs are transcribed leading to production of a miRNA precursor known as the pri-miRNA. The pri-miRNA is typically part of a polycistronic RNA comprising multiple pri-miRNAs. The pri-miRNA may form a hairpin with a stem and loop. The stem may comprise mismatched bases.

In some embodiments, over-expression comprises increasing expression of a naturally expressed miR. In some embodiments, over-expression comprises expression of an exogenous miR. As used herein, an “exogenous miR”, refers to expression of a miR, miR mimic or other synthetic version of the miR that has been introduced into the cell. The cell may express an endogenous form of the miR, but this refers to an externally introduced synthetic form of the miR. In some embodiments, the cell expresses an endogenous form of the exogenous miR. In some embodiments, the cell does not express an endogenous form of the exogenous miR. In some embodiments, the cell is devoid of an endogenous form of the exogenous miR.

The term “microRNA mimic” refers to synthetic non-coding RNAs that are capable of entering the RNAi pathway and regulating gene expression. miRNA mimics imitate the function of endogenous microRNAs (miRNAs) and can be designed as mature, double stranded molecules or mimic precursors (e.g., or pre-miRNAs). miRNA mimics can be comprised of modified or unmodified RNA, DNA, RNA-DNA hybrids, or alternative nucleic acid chemistries (e.g., LNAs or 2′-O, 4′-C-ethylene-bridged nucleic acids (ENA)). Other modifications are described herein below. For mature, double stranded miRNA mimics, the length of the duplex region can vary between 13-33, 18-24 or 21-23 nucleotides. The miRNA may also comprise a total of at least 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39 or 40 nucleotides. The sequence of the miRNA may be the first 13-33 nucleotides of the pre-miRNA. The sequence of the miRNA may also be the last 13-33 nucleotides of the pre-miRNA. The sequence of the miRNA may comprise any of the sequences of the disclosed miRNAs, or variants thereof.

It will be appreciated that the nucleic acid construct of some embodiments of the invention can also utilize miRNA homologues which exhibit the desired activity (i.e., astrocytic differentiating ability). Such homologues can be, for example, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to any of the sequences provided, as determined using the BestFit software of the Wisconsin sequence analysis package, utilizing the Smith and Waterman algorithm, where gap weight equals 50, length weight equals 3, average match equals 10 and average mismatch equals −9.

In addition, the homologues can be, for example, at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to any of the sequences provided herein, as determined using the BestFit software of the Wisconsin sequence analysis package, utilizing the Smith and Waterman algorithm, where gap weight equals 50, length weight equals 3, average match equals 10 and average mismatch equals −9.

The term “expression” as used herein refers to the biosynthesis of a gene product, including the transcription and/or translation of said gene product. Thus, expression of a nucleic acid molecule may refer to transcription of the nucleic acid fragment (e.g., transcription resulting in mRNA or other functional RNA) and/or translation of RNA into a precursor or mature protein (polypeptide). In some embodiments, expression markers refer to RNA expression. In some embodiments, expression markers refer to protein expression.

Introduction of a gene, RNA, nucleic acid or protein into a live cell will be well known to one skilled in the art. As used herein, “introduction” refers to exogenous addition of a gene, miR or compound into a cell. It does not refer to increasing endogenous expression of a gene, protein or compound. Examples of such introduction include, but are not limited to transfection, lentiviral infection, nucleofection, or transduction. In some embodiments, the introduction is by transfection. In some embodiments, the introduction is by lentiviral infection. In some embodiments, the introducing occurs ex vivo. In some embodiments, the introducing occurs in vivo. In some embodiments, the introducing occurs in vivo or ex vivo. In some embodiments, the introduction comprises introducing a vector comprising the gene of interest. In some embodiments, a miR, pre-miR or vector comprising the miR or pre-miR are introduced into the MSC. In some embodiments, the pre-miR is introduced. In some embodiments, the miR is introduced. In some embodiments, a vector comprising the miR, wherein the miR is configured for expression in the MSC is introduced.

The vector may be a DNA plasmid delivered via non-viral methods or via viral methods. The viral vector may be a retroviral vector, a herpesviral vector, an adenoviral vector, an adeno-associated viral vector or a poxviral vector. The promoters may be active in mammalian cells. The promoters may be a viral promoter. The promoter may be a constitutive promoter or an inducible promoter. According to some embodiments, the promoter is a tissue specific promoter.

In some embodiments, the vector is introduced into the cell by standard methods including electroporation (e.g., as described in From et al., Proc. Natl. Acad. Sci. USA 82, 5824 (1985)), Heat shock, infection by viral vectors, high velocity ballistic penetration by small particles with the nucleic acid either within the matrix of small beads or particles, or on the surface (Klein et al., Nature 327. 70-73 (1987)), and/or the like. In some embodiments, the vector, miR, lncRNA or RNA inhibitory molecule are transfected into the MSC.

In some embodiments, down-regulation of expression is achieved by introducing into a cell an inhibitor of the expression. In some embodiments, an inhibitor of expression is selected from a miR, a pre-miR or siRNA. In some embodiments, down-regulation is achieved by genomic alteration such as by CRISPR/cas9 or sleeping beauty technology.

In some embodiments, mammalian expression vectors include, but are not limited to, pcDNA3, pcDNA3.1 (±), pGL3, pZeoSV2(±), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

In some embodiments, expression vectors containing regulatory elements from eukaryotic viruses such as retroviruses are used by the present invention. SV40 vectors include pSVT7 and pMT2. In some embodiments, vectors derived from bovine papilloma virus include pBV-1MTHA, and vectors derived from Epstein Bar virus include pHEBO, and p2O5. Other exemplary vectors include pMSG, pAV009/A+, pMTO10/A+, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

In some embodiments, recombinant viral vectors, which offer advantages such as lateral infection and targeting specificity, are used for in vivo expression. In one embodiment, lateral infection is inherent in the life cycle of, for example, retrovirus and is the process by which a single infected cell produces many progeny virions that bud off and infect neighboring cells. In one embodiment, the result is that a large area becomes rapidly infected, most of which was not initially infected by the original viral particles. In one embodiment, viral vectors are produced that are unable to spread laterally. In one embodiment, this characteristic can be useful if the desired purpose is to introduce a specified gene into only a localized number of targeted cells.

Various methods can be used to introduce the expression vector of the present invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods. It will be appreciated that other than containing the necessary elements for the transcription and translation of the inserted coding sequence (encoding the polypeptide), the expression construct of the present invention can also include sequences engineered to optimize stability, production, purification, yield or activity of the expressed polypeptide.

In some embodiments, introduction of a gene of interest comprises introduction of an inducible vector, wherein administration of a drug to the cell will induce expression of the gene of interest. Drug inducible vectors are well known in the art, some non-limiting examples include tamoxifen-inducible, tetracycline-inducible and doxycycline-inducible. In some embodiments, the inducible-vector is introduced to the MSC ex-vivo and the MSC is contacted with the inducing drug in-vivo. In this way expression of the induced gene, and as a result priming or differentiation of the MSC, only occurs in-vivo. In some embodiments, priming or differentiation of the MSC only occurs after the MSC has homed to a location in the body of a subject.

In some embodiments, introducing comprises introducing a modified RNA. The term “modified RNA” refers to a stable RNA that maybe introduced into the cytoplasm of the cell and will there be translated to protein. Such an RNA does not require transcription for protein expression and thus will more quickly produce protein and is subject to less regulation. Modified RNAs are well known in the art.

During or following the differentiation step the mesenchymal stem cells may be monitored for their differentiation state. Cell differentiation can be determined upon examination of cell or tissue-specific markers which are known to be indicative of differentiation. For example, the differentiated cells may express the following markers: S100 beta, glial fibrillary acidic protein (GFAP), glutamine synthetase, GLT-1, Excitatory Amino Acid Transporter 1 (EAAT1) and Excitatory Amino Acid Transporter 2 (EAAT2). Further, the differentiated cells may secrete a neurotrophic factor including for example glial derived neurotrophic factor (GDNF), GenBank accession nos. L19063, L15306; nerve growth factor (NGF), GenBank accession no. CAA37703; brain-derived neurotrophic factor (BDNF), GenBank accession no CAA62632; neurotrophin-3 (NT-3), GenBank Accession No. M37763; neurotrophin-4/5; Neurturin (NTN), GenBank Accession No. NP-004549; Neurotrophin-4, GenBank Accession No. M86528; Persephin, GenBank accession no. AAC39640; brain derived neurotrophic factor, (BDNF), GenBank accession no. CAA42761; artemin (ART), GenBank accession no. AAD13110; ciliary neurotrophic factor (CNTF), GenBank accession no. NP-000605; insulin growth factor-I (IGF-1), GenBank accession no. NP-000609; and/or Neublastin GenBank accession no. AAD21075. In addition, cell differentiation can be also followed by specific reporters that are tagged with GFP or RFP and exhibit increased fluorescence upon differentiation.

Alternatively, or additionally, the mesenchymal stem cells may be genetically modified so as to express such differentiation factors, using expression constructs such as those described hereinabove.

It will be appreciated that the differentiation time may be selected so as to obtain early progenitors of astrocytes or more mature astrocytes. Enrichment for a particular early or mature astrocytic cell is also contemplated. Selection for cells which express markers such as CD44, A2B5 and S100 allows for the enrichment of progenitor type astrocytes, whereas selection for cells which express markers such as GFAP and glutamine synthase allows for selection of mature astrocytes.

In some embodiments, the differentiation agent is selected from the group consisting of: a growth factor, a lncRNA, a transcription factor and a miR. In some embodiments, the growth factor is selected from the group consisting of: FGF, EGF, PDGF, and FGFbeta and a combination thereof.

### Method of Use

By another aspect, there is provided a method of treating, preventing or ameliorating a neurological disorder, disease or condition in a subject in need thereof, the method comprising administering to a subject a cell of mixed mesenchymal stem cell (MSC) and astrocyte (AS) phenotype (MSC-AS) and/or extracellular vesicles or condition media therefrom.

By another aspect, there is provided a method of treating, preventing or ameliorating a neurological disorder, disease or condition in a subject in need thereof, the method comprising administering to a subject a cell of mixed mesenchymal stem cell (MSC) and astrocyte (AS) phenotype (MSC-AS) and/or extracellular vesicles or condition media therefrom and administering to said subject at least one other cell selected from:
a. an undifferentiated MSC;
b. a glial cell;
c. a neuronal cell; and
d. an MSC transdifferentiated to a neuronal cell;
thereby treating a neurological disorder, disease or condition.

By another aspect, there is provided a method of treating, preventing or ameliorating a neurological disorder, disease or condition in a subject in need thereof, the method comprising administering to a subject an MSC and at least one other cell selected from:
a. a glial cell; and
b. a neuronal cell;
thereby treating a neurological disorder, disease or condition.

In some embodiments, the method comprises administering a pharmaceutical composition comprising the cells. In some embodiments, the cells are the cells of the invention. In some embodiments, the pharmaceutical composition is the pharmaceutical composition of the invention. In some embodiments, the extracellular vesicles are the extracellular vesicles of the invention.

In some embodiments, the different types of cells are administered concomitantly. In some embodiments, the MSC is administered before the other cell. In some embodiments, the MSC-AS is administered before the other cell. In some embodiments, the other cell is administered before the MSC.

In some embodiments, the method is for treating. In some embodiments, the method is for ameliorating. In some embodiments, the method is for preventing. As used herein, the terms “treatment” or “treating” of a disease, disorder, or condition encompasses alleviation of at least one symptom thereof, a reduction in the severity thereof, or inhibition of the progression thereof. Treatment need not mean that the disease, disorder, or condition is totally cured. To be an effective treatment, a useful composition herein needs only to reduce the severity of a disease, disorder, or condition, reduce the severity of symptoms associated therewith, or provide improvement to a patient or subject's quality of life.

The cells and cell populations of the present invention may be useful for a variety of therapeutic purposes. Any disease or disorder with an astrocyte and specifically an A1 astrocyte component may be treated. In some embodiments, the disease, disorder or condition is an A1 astrocyte-associated disease disorder or condition. In some embodiments, the disease, disorder or condition is characterized by A1 astrocyte activity. Representative examples of CNS diseases or disorders that can be beneficially treated with the cells described herein include, but are not limited to, a pain disorder, a motion disorder, a dissociative disorder, a mood disorder, an affective disorder, a neurodegenerative disease or disorder and a convulsive disorder. More specific examples of such conditions include, but are not limited to, Parkinson's disease, Multiple Sclerosis, Huntingdon's disease, myelin relate disorders, leukodystrophy, cerebrovascular disorders, autism spectrum disorders, attention deficit disorders, prior disease, sleep and circadian disorders, neurological inflammation, encephalopathy, Alexander disease, autoimmune encephalomyelitis, diabetic neuropathy, glaucatomus neuropathy, macular degeneration, action tremors and tardive dyskinesia, panic, anxiety, depression, alcoholism, insomnia, manic behavior, Alzheimer's, epilepsy, dementia, vascular dementia, Lewy body dementia prion disorder, Amyotrophic lateral sclerosis (ALS), autism, ischemic brain injury, stroke, Parkinson's disease, multiple system atrophy (MSA), multiple sclerosis (MS), Huntington's disease, demyelination disease, brain injury, spinal injury, concussion, radiation-induce brain injury, epilepsy, aging, neurological aging, chronic pain, infection of the central nervous system (CNS), neuroinflammation and Rett syndrome. In some embodiments, the brain injury is selected from ischemic brain injury and radiation induced brain injury. In some embodiments, the brain injury is white matter injury. In some embodiments, the brain injury is ischemic brain injury. In some embodiments, the brain injury is stroke. In some embodiments, the ischemic brain injury is stroke. In some embodiments, the brain injury is radiation induced brain injury. In some embodiments, the infection is a bacterial infection. In some embodiments, the infection is a viral infection. In some embodiments, the neuroinflammation is neuroinflammation induced by an infection. In some embodiments, the neuroinflammation is induced by sepsis.

In some embodiments, a neurodegenerative disease or condition comprises alpha-synucleinopathies. Non-limiting examples of alpha-synucleinopathies include, but are not limited to Parkinson's disease, multiple system atrophy, and Dementia with Lewy bodies.

In some embodiments, the disease is a disease characterized or caused by alpha-synuclein or elevated levels of alpha-synuclein. In some embodiments, the disease characterized by alpha-synuclein is Parkinson's disease. In some embodiments, the disease is characterized or caused by the presence of Lewy bodies. In some embodiments, the disease is selected from Parkinson's disease, multiple system atrophy and dementia with Lewy bodies. In some embodiments, the disease is selected from multiple system atrophy and dementia with Lewy bodies.

In some embodiments, a neurodegenerative disease or condition comprises any disease or condition comprising the appearance of A1 reactive astrocytes. Methods for identifying A1 astrocytes would be apparent to one of ordinary skill in the art, and can be utilized to detect A1 specific markers, including but are not limited to C3, C4B and CXCL10.

In some embodiments, the neurological disorder, disease or condition is selected from: Alzheimer's disease, depression, a psychiatric disorder, dementia, vascular dementia, Lewy body dementia prion disorder, addiction, withdrawal, substance abuse, Amyotrophic lateral sclerosis (ALS), autism, ischemic brain injury, stroke, Parkinson's disease, multiple system atrophy (MSA), multiple sclerosis (MS), demyelination disease, brain injury, spinal injury, concussion, radiation-induce brain injury, epilepsy, anesthesia-induced cognitive impairment, Huntingdon's disease, myelin relate disorders, leukodystrophy, cerebrovascular disorders, autism spectrum disorders, attention deficit disorders, prior disease, sleep and circadian disorders, neurological inflammation, encephalopathy, Alexander disease, neurological aging, aging, chronic pain, infection of the central nervous system (CNS), neuroinflammation and Rett syndrome. In some embodiments, the neurological disorder, disease or condition is one characterized by astrocyte involvement. In some embodiments, the neurological disorder, disease is characterized by A1 astrocytes. In some embodiments, the neurological disorder, disease is an astrocyte, or A1 astrocyte related disease, disorder or condition. In some embodiments, the neurological condition is neurological damage and/or neurological injury. In some embodiments, the neurological disease is ALS. In some embodiments, the neurological disease is Rett syndrome. In some embodiments, the neurological disease is selected from ALS and Rett syndrome. In some embodiments, the brain injury is selected from ischemic brain injury, stroke and radiation induced brain injury. In some embodiments, the brain injury is white matter injury. In some embodiments, the neurological disease is Parkinson's disease. In some embodiments, the neurological disease is brain damage. In some embodiments, the neurological disease is brain injury. In some embodiments, the brain injury is radiation induced injury. In some embodiments, the brain injury is white matter injury. In some embodiments, the brain injury is ischemic brain injury. In some embodiments, the brain injury is recurrent brain injury. In some embodiments, the brain injury is traumatic brain injury. In some embodiments, the brain injury is concussion. In some embodiments, the brain injury is prolonged brain injury. In some embodiments, the prolonged injury is prolonged ischemia. In some embodiments, the radiation induced injury is acute radiation induced injury. In some embodiments, prolonged injury is spaceflight. In some embodiments, the neurological disease, disorder or condition is selected from ALS, Parkinson's disease, and brain injury. In some embodiments, the neurological disease is chronic pain.

The use of differentiated MSCs may be also indicated for treatment of traumatic lesions of the nervous system including spinal cord injury and also for treatment of stroke caused by bleeding or thrombosis or embolism because of the need to induce neurogenesis and provide survival factors to minimize insult to damaged neurons.

The cells of the present invention can be administered to the treated individual using a variety of transplantation approaches, the nature of which depends on the site of implantation.

The term or phrase “transplantation”, “cell replacement” or “grafting” are used interchangeably herein and refer to the introduction of the cells of the present invention to target tissue. As mentioned, the cells can be derived from the recipient or from an allogeneic, semi-allogeneic or xenogeneic donor.

By another aspect, there is provided a method of increasing engraftment of cells into a subject in need thereof, the method comprising co-administering with the cells any one of: a pharmaceutical composition of the invention, a pharmaceutical composition comprising unmodified MSCs, and a combination thereof, thereby increasing engraftment of the cells.

By another aspect, there is provided a composition comprising any one of: a cell of mixed character of the invention, an unmodified MSC, and a combination thereof, for use in increasing engraftment of cells.

By another aspect, there is provided a composition comprising any one of: a cell of mixed character of the invention, an unmodified MSC, and a combination thereof, for use in treating, preventing or ameliorating a neurological disorder, disease or condition in a subject in need thereof.

According to some embodiments, the cells can be injected systemically into the circulation, administered intrathecally or grafted into the central nervous system, the spinal cord or into the ventricular cavities or subdurally onto the surface of a host brain. Conditions for successful transplantation include: (i) viability of the implant; (ii) retention of the graft at the site of transplantation; and (iii) minimum amount of pathological reaction at the site of transplantation. Methods for transplanting various nerve tissues, for example embryonic brain tissue, into host brains have been described in: “Neural grafting in the mammalian CNS”, Bjorklund and Stenevi, eds. (1985); Freed et al., 2001; Olanow et al., 2003). These procedures include intraparenchymal transplantation, i.e. within the host brain (as compared to outside the brain or extraparenchymal transplantation) achieved by injection or deposition of tissue within the brain parenchyma at the time of transplantation.

According to some embodiments, intraparenchymal transplantation can be performed using two approaches: (i) injection of cells into the host brain parenchyma or (ii) preparing a cavity by surgical means to expose the host brain parenchyma and then depositing the graft into the cavity. Both methods provide parenchymal deposition between the graft and host brain tissue at the time of grafting, and both facilitate anatomical integration between the graft and host brain tissue. This is of importance if it is required that the graft becomes an integral part of the host brain and survives for the life of the host.

According to some embodiments, the graft may be placed in a ventricle, e.g. a cerebral ventricle or subdurally, i.e. on the surface of the host brain where it is separated from the host brain parenchyma by the intervening pia mater or arachnoid and pia mater. Grafting to the ventricle may be accomplished by injection of the donor cells or by growing the cells in a substrate such as 3% collagen to form a plug of solid tissue which may then be implanted into the ventricle to prevent dislocation of the graft. For subdural grafting, the cells may be injected around the surface of the brain after making a slit in the dura.

According to some embodiments, injections into selected regions of the host brain may be made by drilling a hole and piercing the dura to permit the needle of a microsyringe to be inserted. The microsyringe is preferably mounted in a stereotaxic frame and three-dimensional stereotaxic coordinates are selected for placing the needle into the desired location of the brain or spinal cord. The cells may also be introduced into the putamen, nucleus basalis, hippocampus cortex, striatum, substantia nigra or caudate regions of the brain, as well as the spinal cord.

According to some embodiments, the cells may also be transplanted to a healthy region of the tissue. In some cases, the exact location of the damaged tissue area may be unknown, and the cells may be inadvertently transplanted to a healthy region. In other cases, it may be preferable to administer the cells to a healthy region, thereby avoiding any further damage to that region. Whatever the case, following transplantation, the cells preferably migrate to the damaged area.

According to some embodiments, for transplanting, the cell suspension is drawn up into the syringe and administered to anesthetized transplantation recipients. Multiple injections may be made using this procedure.

According to some embodiments, the cellular suspension procedure permits grafting of the cells to any predetermined site in the brain or spinal cord, is relatively non-traumatic, allows multiple grafting simultaneously in several different sites or the same site using the same cell suspension, and permits mixtures of cells from different anatomical regions.

According to some embodiments, multiple grafts may consist of a mixture of cell types, and/or a mixture of transgenes inserted into the cells. Preferably from approximately 10⁴ to approximately 10⁹ cells are introduced per graft. Cells can be administered concomitantly to different locations such as combined administration intrathecally and intravenously to maximize the chance of targeting into affected areas.

According to some embodiments, for transplantation into cavities, which may be preferred for spinal cord grafting, tissue is removed from regions close to the external surface of the central nerve system (CNS) to form a transplantation cavity, for example as described by Stenevi et al. (Brain Res. 114:1-20, 1976), by removing bone overlying the brain and stopping bleeding with a material such a gelfoam. Suction may be used to create the cavity. The graft is then placed in the cavity. More than one transplant may be placed in the same cavity using injection of cells or solid tissue implants. Preferably, the site of implantation is dictated by the CNS disorder being treated. Demyelinated MS lesions are distributed across multiple locations throughout the CNS, such that effective treatment of MS may rely more on the migratory ability of the cells to the appropriate target sites.

Intranasal administration of the cells described herein is also contemplated.

According to some embodiments, since non-autologous cells may induce an immune reaction when administered to the body the cells may be administered to privileged sites, or alternatively, the recipient's immune system may be suppressed by providing anti-inflammatory treatment which may be indicated to control autoimmune disorders to start with and/or encapsulating the non-autologous/semi-autologous cells in immunoisolating, semipermeable membranes before transplantation. This may not be necessary as the cells of the invention does not induce immune response and/or suppress immune response.

The cells of the present invention may be co-administered with therapeutic agents useful in treating neurodegenerative disorders, such as gangliosides; antibiotics, neurotransmitters, neurohormones, toxins, neurite promoting molecules; and antimetabolites and precursors of neurotransmitter molecules such as L-DOPA.

As used herein, the term “about” when combined with a value refers to plus and minus 10% of the reference value. For example, a length of about 1000 nanometers (nm) refers to a length of 1000 nm+−100 nm.

It is noted that as used herein and in the appended claims, the singular forms “a,” “an,” and “the” include plural referents unless the context clearly dictates otherwise. Thus, for example, reference to “a polynucleotide” includes a plurality of such polynucleotides and reference to “the polypeptide” includes reference to one or more polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements or use of a “negative” limitation.

In those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the invention are specifically embraced by the present invention and are disclosed herein just as if each and every combination was individually and explicitly disclosed. In addition, all sub-combinations of the various embodiments and elements thereof are also specifically embraced by the present invention and are disclosed herein just as if each and every such sub-combination was individually and explicitly disclosed herein.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

Various embodiments and aspects of the present invention as delineated hereinabove and as claimed in the claims section below find experimental support in the following examples.

### EXAMPLES

Generally, the nomenclature used herein, and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Strategies for Protein Purification and Characterization—A Laboratory Course Manual” CSHL Press (1996); all of which are incorporated by reference. Other general references are provided throughout this document.

Materials and Methods:

Methods of Transdifferentiating MSCs to Astrocytes:

Protocol 1. MSCs were maintained in NSC medium containing neurobasal medium supplemented with 2% B27 and CHIR99021 (2 mM) in low-attachment plates for a week. Medium was then supplemented with 0.2 mM retinoic acid, dcAMP, purmorphamine, bFGF and FGF2. After 10 days, the cells were treated with LIF, and BMP4 (10 ng/ml each) for an additional 7-10 days. At the end of the incubation cells expressed GFAP and EAAT2 (>95%) and high levels of BDNF and GDNF. MSCs may first be transdifferentiated to NSCs before beginning the protocol.

Protocol 2. MSCs are exposed to pH 6.0 for an hour, followed by hypoxia overnight and then maintained in F12+B27 with FGF and EGF for a week in low attachment plates. The cells are then transferred to DMEM+serum, a beta-adrenergic receptor agonist (albuterol), PDGF (5 ng/ml), neuregulin (50 ng/ml) and FGFbeta (10 ng/ml) for a week. The cells are then transferred to DMEM+G5 supplemented medium together with albuterol, neuregulin and FGF for another week. MSCs may first be transdifferentiated to NSCs before beginning the protocol.

Methods of Transdifferentiating MSCs to NSCs:

Protocol 3. MSC are exposed to pH 6.0, for 1 hr followed by treatment with a Rock inhibitor, and hypoxia overnight and then maintained in DMEM+B27+N2+EGF and FGF (10 ng/ml each) in low attachment plates. Cells are maintained as spheroids.

Protocol 4. The same as protocol 3, but 10 μM SB431542 and 100 ng/mL Noggin are also added to the DMEM.

Protocol 5. The same as protocol 4, but SOX2 and/or BRN2 are overexpressed in the cell.

A1/A2 ratio measurements: Human astrocytes were stimulated with a combination of C1q, IL-1 and TNF-alpha or co-cultured with LPS-stimulated human microglia cells for the generation of A1 astrocytes or with IL-4 for the generation of A2. The expression of the A1 markers C3 and that of S100A10 were determined and the ratio between them was designated the A1/A2 ratio. This was set as 1 for each of the phenotypes. The relative A1/A2 was determined for MSC-AS or Astrocytes that were stimulated similarly to the description provided herein in the presence of MSC, MSC-AS or their exosomes.

### Example 1: MSC-AS Produced by New Protocols are Protected from the A1 Phenotype

MSC-AS cells were produced by Protocol 1 (see Materials and Methods) and their expression profile was assessed by RT-PCR. The cells highly expressed astrocyte markers GFAP, EAAT1 and EAAT2, and secreted neurotrophic factors including BDNF, GDNF, Neurturin, NGF, NT-3 and VEGF. Additionally, the cells continued to express many MSC markers including a lack of MHCII and secretion of IL-4 and IL-10. Unexpectedly, the cells also expressed a number of astrocyte genes whose expression had never been reported when MSC were transdifferentiated. This included S100A10, TGM1, PTX3, SPHK1, CD109, Arginase-1, TM4SFL, S1PR3, CLCF1, LCN2, NRF2, prokineticin-2, STAT3 and PKC epsilon. Expression of GFAP, S100b, EAAT1 and EAAT2 was also seen as has been previously reported. Many of these proteins are more highly expressed in A2 astrocytes as compared to A1 astrocytes. As A1 astrocytes are generally considered toxic and A2 astrocytes protective, MSC-AS with an increased A2 phenotype were hypothesized to be therapeutically advantageous. Similar gene expression results were observed when protocol 2 was employed.

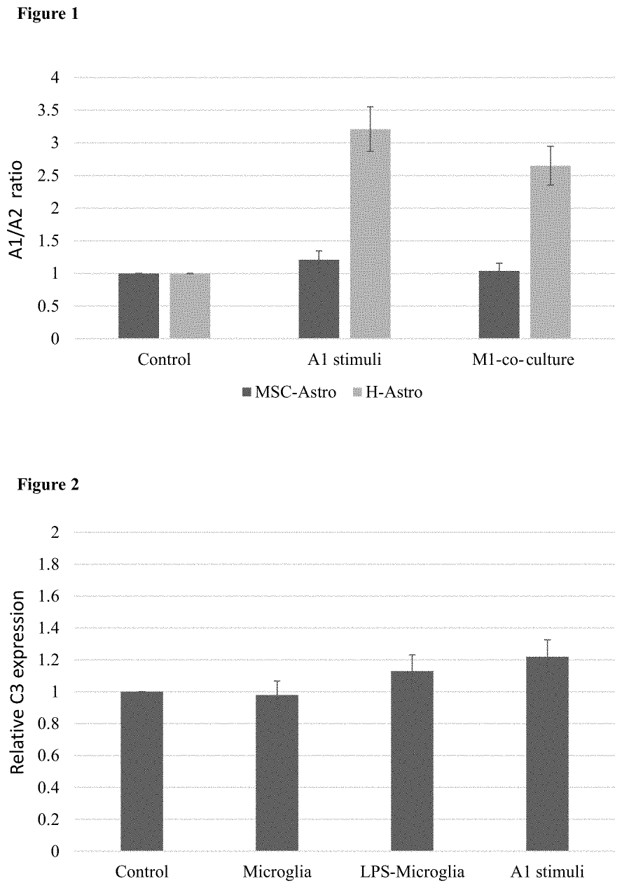

Not only did these new MSC-AS cells have increased A2 gene expression, but the cells were also inhibited from acquiring the A1 phenotype. MSC-AS and naturally occurring human AS were separately treated with stimuli that are known to induce an A1 phenotype. Specifically, the cells were incubated with A1 stimuli (IL-1 alpha (3 ng/ml); TNF-alpha (30 ng/ml) and C1q (400 ng/ml)) or cocultured in a transwell plate with microglia stimulated with LPS (100 ng/ml) for 48 hr and A1 to A2 ratio was determined. As can be seen in FIG. 1, both types of A1 stimulation induced a strong induction of A1 phenotype in the natural AS, with the ratio of A1 to A2 tripling on average. By contrast, the MSC-AS cells were highly resistant to A1 induction, with the first stimulation having a very minor, non-significant effect and with the microglia having no effect at all. This is on top of the fact that MSC-ACs already are shifted toward an A2 phenotype (both controls were standardized to 1, even though MSC-AS showed stronger expression of A2 markers).

To reinforce the anti-A1 effect in these MSC-AC, the A1 marker C3 was examined. MSC-AS were cultured in a transwell dish either alone, with microglia, with microglia stimulated with LPS or with A1 stimuli (as above) and then C3 mRNA expression was examined by quantitative PCR. As can be seen in FIG. 2, C3 expression was virtually unaffected in these cells. This data strongly suggests that the MSC-AS of the invention are blocked or inhibited from acquiring the A1 phenotype.

### Example 2: MSC and their Exosomes Protected Other Cells from Acquiring the A1 Phenotype

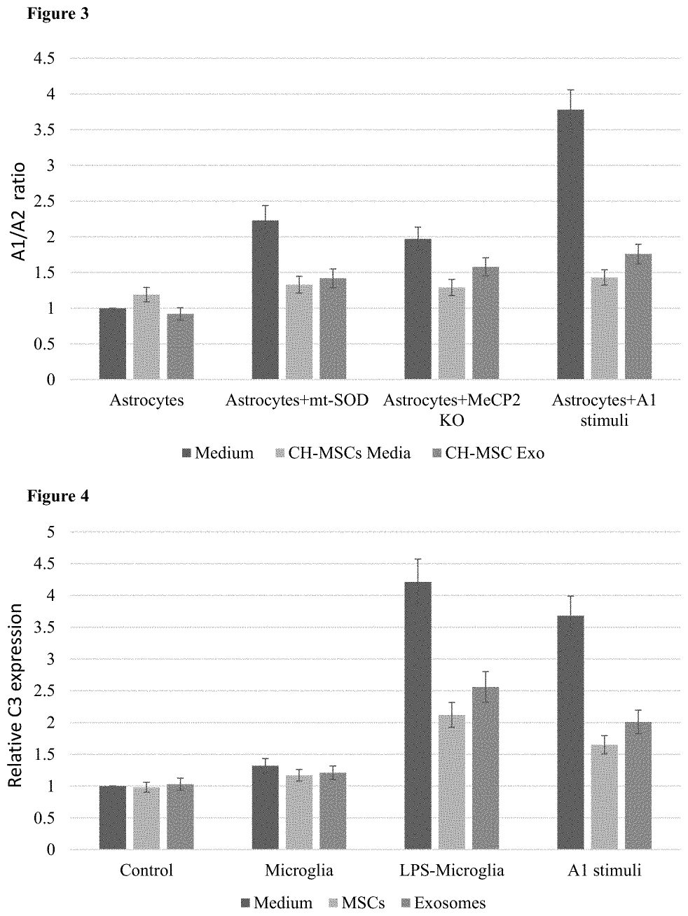

Astonishingly, not only were the MSC-AS cells produced by Protocols 1 and 2 themselves protected from A1 induction, but MSCs in general (unmodified) and the secreted vesicles from MSCs and MSC-ASs also protected other cells from induction. The A1/A2 ratio was measured in human astrocytes expressing a control vector, expressing a SOD mutant expression vector (G93A mutation, which is a model for ALS), silenced for MeCP2 (which is a model for Rett Syndrome) or cultured with A1 stimuli (as before). In culture alone, SOD1 mutant expression and silencing of MeCP2 induced a doubling of the A1/A2 ratio, while A1 stimuli, as seen in FIG. 1, produced a greater than 3-fold increase (FIG. 3). When these cells were cultured, in the presence of conditioned media from undifferentiated chorionic MSCs (CH-MSC), or the MSCs themselves in a transwell dish, the A1/A2 ratio was barely increased over WT control astrocytes. Similarly, when exosomes isolated from undifferentiated CH-MSCs were added the same inhibition was observed.

To reinforce this point, the A1 marker C3 was examined as before. Natural human astrocytes were cultured in a transwell dish either alone, with microglia, microglia stimulated with LPS, or A1 stimuli. Incubation with LPS-microglia or A1 stimuli greatly increased C3 expression as expected (FIG. 4). By contrast, when undifferentiated MSCs or their exosomes were added to the culture the C3 levels in the astrocytes were significantly reduced (FIG. 4). This data shows that undifferentiated MSCs have a protective effect on astrocytes and inhibit A1 conversion. Further, this effect is mediated by secreted factors including extracellular vesicles and does not require direct cell contact. When UC-MSCs, their media, or exosomes were used similar results were observed. Similarly, when MSC-AS, their media, or exosomes were used similar results were observed.

### Example 3: MSC-AS Replacement Therapy for ALS Treatment

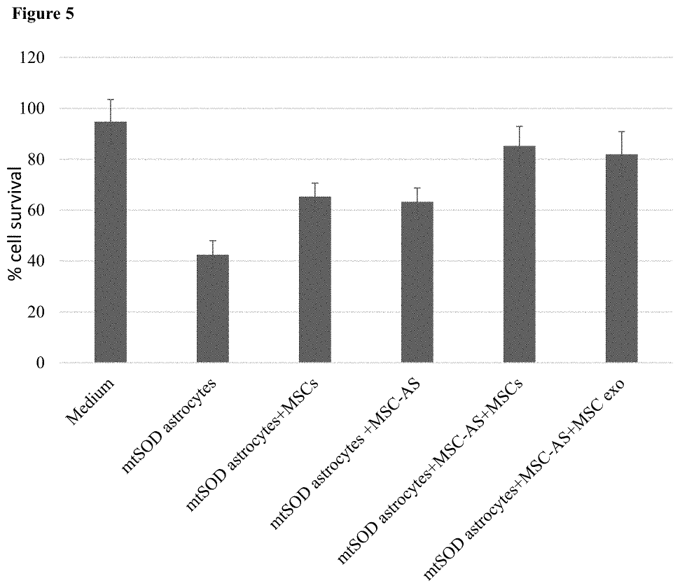

In order to test if MSC-AS cell replacement therapy could be therapeutically viable for ALS treatment, motor neuron survival was examined in the presence of mutant SOD1 (mtSOD) expressing astrocytes with and without various MSC combinations. Firstly, human motor neurons were cocultured in a transwell dish with mtSOD expressing astrocytes and neuron survival was measured by XTT. After 10 days, survival had dropped by more than 55% percent (FIG. 5). In contrast, when the coculture included undifferentiated MSCs or MSC-AS along with the mtSOD astrocytes, a 50% improvement was observed. When the coculture included both MSC-AS and undifferentiated MSCs or their exosomes, survival of the neurons doubled as compared to the culture with mtSOD astrocytes alone. This data shows that MSCs exert a protective effect on motor neurons in an ALS model, and that combination of MSCs (or their exosomes) with MSC-AS cells has an enhanced effect.

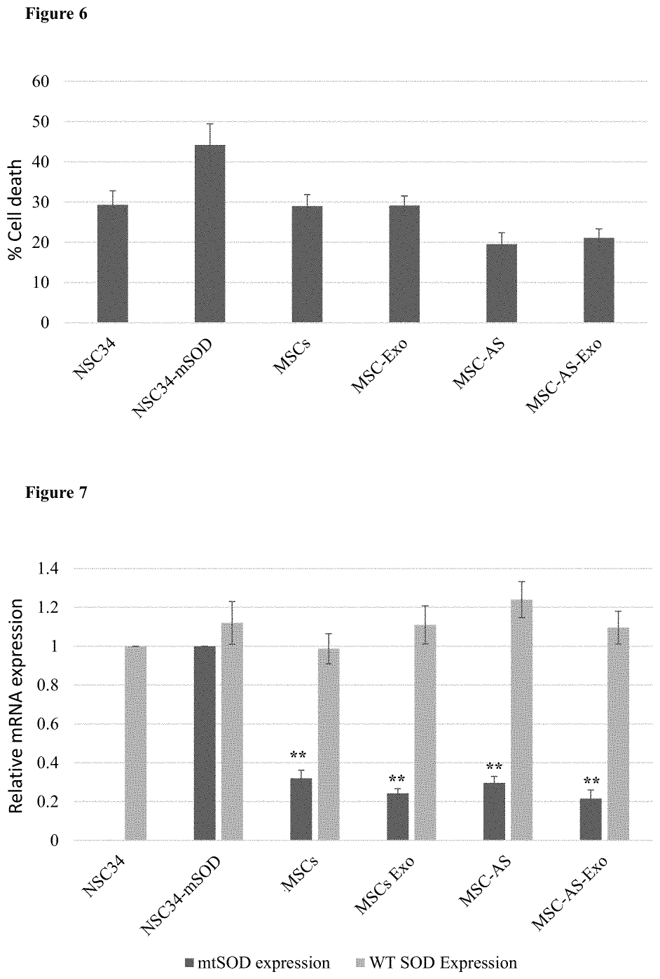

To further test efficacy in treating ALS, cells of the NSC34 motor neuron cell line were co-cultured with astrocytes comprising mtSOD in a transwell dish. Cell death events were measured in the NSC34 cells. When the NSC34 cells also expressed mtSOD cell death was increased in the co-cultured cells as compared to wild-type NSC34 cells, however, addition of MSCs (both CH and UC produced similar results), MSC-AS or their exosomes to the culture alleviated this increased cell death (FIG. 6). Indeed, the MSC-AS and their exosomes actually decreased cell death below the baseline of the NSC34 cells grown alone.

To test if differentiated MSCs and their exosomes can be used to carry therapeutics, the same experimental set up with mtSOD expressing NSC34 cells was employed. In this experiment the MSCs and MSC-AS cells expressed an anti-sense oligonucleotide (ASO) specific to the mutant SOD. After the co-culture WT and mutant SOD protein was measured by immunoblot. The MSCs (both CH and UC MSCs showed similar results) and their exosomes effectively transfer the ASO to the NSC34 cells as mutant protein levels were decreased by over 70% (FIG. 7). The results demonstrate that both control and differentiated cells and their EVs can similarly deliver the ASO to the NSC34 cells. This reinforces that RNA therapeutics can be transferred in extracellular vesicles. Further, regardless of what cells and exosomes were used, WT SOD protein levels stayed constant. Thus, MSCs, MSC-AS and their exosomes are effective carriers for other therapeutics as well.

Based on these results it is apparent that using MSCs or exosomes that exert therapeutic effects on a specific disease can be superior for the delivery of RNA, peptide-based or other therapies, compared with MSCs and exosomes that do not exert such effects. Thus, bioinformatics tools or experimental data is used to identify specific MSCs or exosomes, either modified or unmodified, that exert therapeutic effect in a specific disease on their own, and those specific cells or exosomes are employed as a delivery tool for the treatment of that disease. Moreover, bioinformatics analysis or experimental data is also used to determine maximal synergistic effects of these “therapeutic MSCs and/or exosomes” and the therapies that they deliver in order to maximize the effects. This concept is exemplified by the administration of MSC-AS and AS to mutant SOD but can be extended to the combinations of other cells and exosomes and specific disease-related therapies. In essence personalized cells and exosomes are used as delivery tools and personalized delivered therapy for the treatment of diseases.

### Example 4: MSCs, MSC-AS and their Extracellular Vesicles for Treating Parkinson's Disease

Having shown a therapeutic effect in ALS, the utility of these cells and their vesicles was tested in Parkinson's disease. Mice were injected with 6-hydroxy dopamine in the right striatum to model Parkinson's disease. One day later CH-MSC, their vesicles, MSC-AS, their vesicles or PBS were administered intranasally. 200,000 cells/3 ul of each of the cells was administered in PBS, and 1×10\hat{ }8 extracellular vesicles were administered in PBS. Media alone was used as a control. At day 28 after the cell injections D-amphetamine induced rotational behavior was measured as an output for Parkinson's severity. Mice that received only PBS showed severe impairment with 3.76±0.86 ipsilateral rotations per min. CH-MSCs decreased the number of rotations to 2.21±0.63, while their vesicles produced a decrease to 2.38±0.71 ipsilateral rotations per min. Even more impressive, MSC-AS produced a reduction to 1.64±0.51, while their extracellular vesicles produced a reduction to 1.78±0.68. All of these reductions were statistically significant, and similar results were observed with UC-MSCs and their vesicles.

### Example 5: MSC and their Extracellular Vesicles for Treating Brain Injury

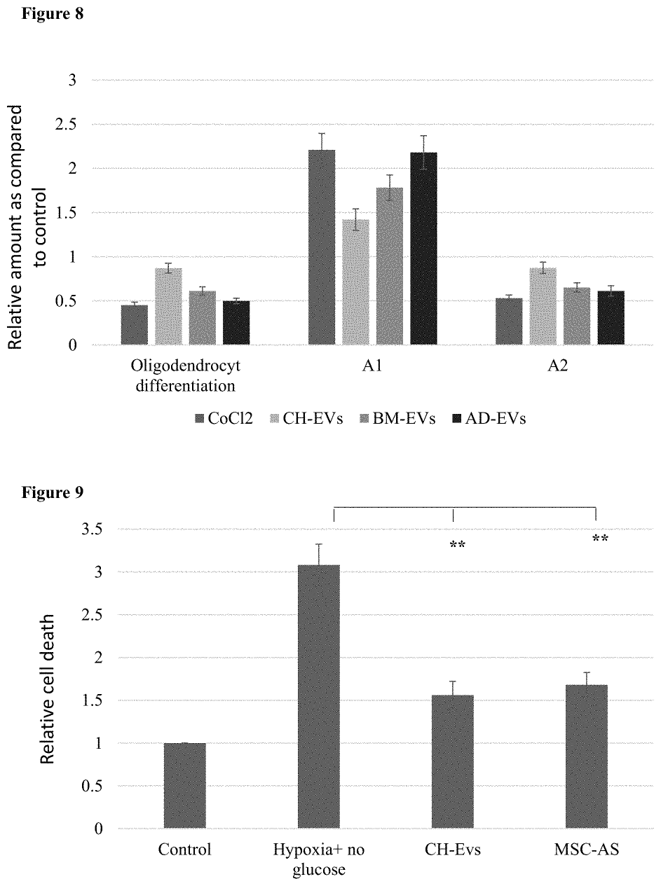

In order to test if MSCs can improve the prognosis of brain injury, and/or prevent them, oligodendrocyte differentiation was monitored as a model for white matter injury. Astrocytes and oligodendrocyte progenitor cells (OPCs) were cocultured in oligodendrocyte differentiation medium with and without the presence of CoCl2 (5 uM). CoCl2 induces astrocyte conversion from A2 to A1 astrocytes and mimics the effect of brain injury. The level of oligodendrocyte differentiation was determined by the expression of the marker MBP, and the presence of A1 and A2 astrocytes were determined by expression of Csd and S100A10 respectively. Addition of CoCl2 to the culture increased the number of A1 astrocytes, decreased the number of A2 astrocyte and reduced the amount of oligodendrocyte differentiation by 55% as expected (FIG. 8, all amounts are relative to levels in control coculture without CoCl2). Addition of extracellular vesicles from undifferentiated CH-MSCs had a protective effect on the coculture (FIG. 8): oligodendrocyte differentiation was doubled (˜100% increase), and A1/A2 ratio was greatly improved. Bone marrow (BM) derived MSCs had a similar, though reduced effect, with oligodendrocyte differentiation increasing by only ˜35%. Adipose (AD) derived MSCs had almost no effect.

Next a model of ischemic brain injury was tested. A cell line of human neurons (NT-2) were cultured in a transwell setup with astrocytes cultured on the other side of the transwell. The plate was incubated in hypoxic conditions (5% CO₂ and 95% N2) and without glucose for 6 hours, followed by 42 hours of culture in standard conditions (normoxia, normal glucose). Cell death was monitored at the end of the 48 hours, and the pseudo-ischemic conditions produced more than 3-times the cell death as compared to control cells kept in standard conditions (FIG. 9). In contrast, when extracellular vesicles from CH-MSC were added to the culture the amount of induced cell death was halved. Similarly, when MSC-AS were used for the transwell culture in place of the natural astrocytes a similar reduction in cell death was observed. Both reductions were statistically significant, and similar results were observed with UC-MSCs. This shows that administering MSCs, their vesicles, MSC-AS or their vesicles are all effective in protecting neurons from ischemic conditions, and thus are effective treatments for ischemic brain injury.

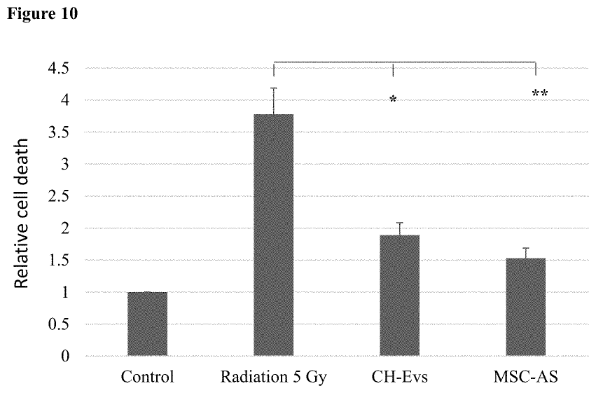

A similar setup was used to test radiation-induced brain injury. Motor neurons were transwell cultured with astrocytes, with and without extracellular vesicles from CH-MSC, or with MSC-AS, as before. Instead of ischemic conditions, the transwell culture was irradiated with 5 grays of radiation and cell death was quantified after 48 hours (FIG. 10). Irradiation increased cell death by more than 3.5 times as compared to the unirradiated control. As with ischemic brain injury, vesicles from CH-MSC resulted in a significant reduction in cell death of −50%. MSC-AS cells were even more effective, reducing cell death by 60%. Taken together this data shows that MSCs, their vesicles, MSC-AS and their vesicles are all suitable for treatment of a wide variety of brain injuries.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1-33. (canceled)

34. A cell comprising mixed mesenchymal stem cell (MSC) and astrocyte (AS) phenotypes (MSC-AS), wherein said cell expresses at least one marker selected from: S100A10, TGM1, PTX3, SPHK1, CD109, Arginase-1, TM4SFL, S1PR3, CLCF1, LCN2, NRF2, prokineticin-2, STAT3 and PKC epsilon.

35. The cell of claim 34, wherein said astrocyte phenotype is an A2 astrocyte phenotype.

36. The cell of claim 34, wherein said cell is resistant to induction to an A1 astrocyte phenotype or inhibits the differentiation of astrocytes toward an A1 phenotype, optionally wherein said induction comprises stimulation with at least one of C1q, IL-1, TNF-alpha and LPS-induced microglial cells.

37. The cell of claim 34, wherein said cell comprises an MSC phenotype comprising at least one of:
a. expression of a plurality of markers selected from the group consisting of: CD73, CD105, CD90, CD146, and CD44 expression and absence of WWII expression;
b. immunosuppression ability;
c. anti-inflammatory ability;
d. the ability to home to sites of inflammation, injury or disease, and
e. expression and/or secretion of neurotrophic factors.

38. A method of producing a cell of mixed MSC and AS phenotypes (MSC-AS), the method comprising at least one of:
a. incubating an MSC or MSC transdifferentiated into a neuronal stem cell (NSC) in low-attachment plates in a first medium and inhibiting GSK3 in said MSC or transdifferentiated MSC; further incubating in a second medium supplemented with retinoic acid, a cAMP activator, and a hedgehog activator; and further incubating in a third medium supplemented with leukemia inhibitory factor (LIF), and Bone morphogenetic protein-4 (BMP4); and
b. incubating an MSC in a first medium supplemented with growth factors in low-attachment plates; further incubating in a second medium comprising serum supplemented with a beta-adrenergic receptor agonist, a neuregulin and growth factors and further incubating in a third medium supplemented with G5, a beta-adrenergic receptor agonist, a neuregulin and growth factors;
thereby producing a hybrid MSC-AS cell.

39. The method of claim 38, wherein at least one of SOX2 and BRN2 is overexpressed in said MSC transdifferentiated to an NSC before said incubating in a first media.

40. The method of claim 38, wherein said first media is neurobasal medium or F12 media supplemented with B27, said second media further comprises growth factors, or both, optionally wherein said growth factors are selected from FGF, EGF, PDGF, and FGFbeta.

41. The method of claim 38, further comprising selecting a cell that expresses EAAT1 and/or EAAT2 or secretes a neurotrophic factor selected from BDNF, GDNF, Neurturin, NGF, NT-3, and VEGF.

42. The method of claim 38, further comprising at least one of:
a. expressing in said MSC or transdifferentiated MSC at least one of: SOX9, NF1A, NF1B, STAT3, miR-21, miR-27, miR-152, miR-455, miR-203, miR-355, let-7, and miR-1;
b. inhibiting in said MSC or transdifferentiated MSC at least one of: miR-224, miR-3191, miR-124, miR-145, miR-1277, miR-107, miR-130, miR-190, miR-1277, miR-190, miR-19, miR-331, combination of miR-124, miR-145 and miR-1277, miR-223, miR-3714, miR-3924, miR-5011, miR-6801, miR-1224, miR-1305, miR-3153, and miR-137; optionally wherein said inhibiting comprises expressing in said MSC or transdifferentiated MSC an RNA that hybridizes to and inhibits said miR; and
c. inhibiting in said MSC or transdifferentiated MSC at least one of: SNAIL TWIST1, RUNX2 and SOX11.

43. A cell produced by the method of claim 38.

44. Extracellular vesicles from a cell of claim 34.

45. A pharmaceutical composition comprising at least one of:
a. a cell of claim 34;
b. extracellular vesicles from a cell of claim 34; and
c. conditioned media from a cell of claim 34;
and a pharmaceutically acceptable carrier, excipient or adjuvant.

46. The pharmaceutical composition of claim 45 and at least one of:
a. an undifferentiated MSC;
b. a natural glial cell;
c. a natural neuronal cell;
d. an MSC transdifferentiated to a neuronal cell; and
e. exosomes, extracellular vesicles or conditioned media therefrom.

47. The pharmaceutical composition of claim 46, wherein said natural neuronal cell is an NSC, said natural glial cell is an astrocyte or both.

48. A method of treating a neurological disorder, disease or condition, in a subject in need thereof, the method comprising administering to said subject at least one of:
a. a cell of mixed mesenchymal stem cell (MSC) and astrocyte (AS) phenotype (MSC-AS);
b. exosomes, extracellular vesicles or condition media from said MSC-AS;
c. a chorionic placenta (CH) or umbilical cord (UC) derived MSC; and
d. exosomes, extracellular vesicles or condition media from said CH or UC derived MSC;
thereby treating a neurological disorder, disease or condition.

49. The method of claim 48, further comprising administering to said subject at least one other cell selected from:
a. an undifferentiated MSC;
b. a natural glial cell;
c. a natural neuronal cell; and
d. an MSC transdifferentiated to a neuronal cell.

50. The method of claim 48, comprising administering a pharmaceutical composition comprising a cell comprising mixed mesenchymal stem cell (MSC) and astrocyte (AS) phenotypes (MSC-AS), wherein said cell expresses at least one marker selected from: S100A10, TGM1, PTX3, SPHK1, CD109, Arginase-1, TM4SFL, S1PR3, CLCF1, LCN2, NRF2, prokineticin-2, STAT3 and PKC epsilon or exosomes, extracellular vesicles or condition media therefrom and a pharmaceutically acceptable carrier, excipient or adjuvant.

51. The method of claim 48, wherein said MSC-AS, CH MSC, UC MSC or exosomes, extracellular vesicles or condition media therefrom is administered concomitantly, before or after said at least one other cell.

52. The method of claim 48, comprising administering said MSC-AS or exosomes, extracellular vesicles or condition media from said MSC-AS.

53. The method of claim 48, wherein said neurological disorder, disease or condition is selected from: Alzheimer's disease, depression, a psychiatric disorder, dementia, vascular dementia, Lewy body dementia prion disorder, addiction, withdrawal, substance abuse, Amyotrophic lateral sclerosis (ALS), autism, ischemic brain injury, stroke, Parkinson's disease, multiple system atrophy (MSA), multiple sclerosis (MS), Huntingdon's disease, myelin relate disorders, leukodystrophy, cerebrovascular disorders, autism spectrum disorders, attention deficit disorders, prior disease, sleep and circadian disorders, neurological inflammation, encephalopathy, Alexander disease, demyelination disease, brain injury, spinal injury, concussion, radiation-induce brain injury, epilepsy, anesthesia-induced cognitive impairment, aging, neurological aging, chronic pain, infection of the central nervous system (CNS), neuroinflammation and Rett syndrome, optionally wherein said neurological disorder, disease or condition is selected ALS, Parkinson's disease, brain injury, radiation-induced brain injury and ischemic brain injury; said brain injury is selected from traumatic brain injury, stroke, radiation-induced brain injury, ischemic brain injury, prolonged ischemic brain injury, acute radiation induced brain injury, concussion and spaceflight induced brain injury or both.
